(19) United States
(12) Patent Application Publication
(10) Pub. No.: US 2004/0002457 A1
(43) Pub. Date: Jan. 1, 2004

(54) Inhibition of virus anchorage by RGG domain of a cell surface-expressed protein, polynucleotide coding for said RGG domain, therapeutic uses thereof by inhibition of microorganism or protein ligand binding to the cell-surface-expressed protein

(76) Inventors: Ara V. Hovanessian, Bourg la Reine (FR); Jean-Paul Briand, Strasbourg (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/384,569

(22) Filed: Mar. 11, 2003

Related U.S. Application Data
(60) Provisional application No. 60/363,371, filed on Mar. 12, 2002.
(60) Provisional application No. 60/397,600, filed on Jul. 23, 2002.

(51) Int. Cl. A61K 38/17; C07K 14/47

(52) U.S. Cl. 514/12; 530/350

(57) ABSTRACT

Peptides that are involved in the attachment of a microorganism or a protein ligand to the cell membrane are provided. The peptides comprise one RGG domain of a cell-surface expressed protein. These peptides include peptides from nucleolin, specifically the C-terminal portion, and more specifically the last 63 amino acids of the C-terminal portion. These peptides can inhibit binding of viruses, such as HIV, to the cell membrane. Also provided is a therapeutic composition of these peptides and a method of treating an infection by a micrioorganism using these peptides.

### CROSS-REFERENCE TO RELATED APPLICATIONS

This application claims the benefit of U.S. Provisional Patent Application No. 60/363,371, filed Mar. 12, 2002, and U.S. Provisional Patent Application No. 60/397,600, filed Jul. 23, 2002, the entire disclosures of each of which are incorporated herein in their entirety for all purposes.

### BACKGROUND OF THE INVENTION

HIV infects target cells by the capacity of its envelope glycoproteins, the gp120-gp41 complex, to attach cells and induce the fusion of virus and cell membranes. The receptor complex for HIV entry consists of the CD4 molecule and at least one of the members of the chemokine receptor family; CCR5 is the major coreceptor for macrophage-tropic HIV-1 isolates (R5), whereas that for T-lymphocyte-tropic isolates (X4) is CXCR4 (1). Although both CD4 and CXCR4/CCR5 are essential for the HIV entry process, the initial association of HIV particles to cells (referred to as attachment) could occur in the absence or blockade of these receptors (2-4). Accordingly, HIV attachment occurs efficiently in CD4-human cells and even in heterologous cells albeit in the absence of membrane fusion and viral entry (4, 5). Several observations have pointed out that attachment of HIV particles to the cell surface seems to occur through the coordinated interactions on the one hand with heparan sulfate proteoglycans (2, 6, 7) and on the other hand with the cell-surface-expressed nucleolin (3, 4). Consequently, targeting any one of these components could result in the inhibition of HIV attachment. Indeed, HIV attachment could be blocked either by the fibroblast growth factor 2 (FGF-2) that binds heparan sulfate proteoglycans or by the anti-HIV pseudopeptide HB-19 which binds nucleolin (3, 4). Contradictory hypothesis has been provided for the interaction of HIV particles with heparan sulfate proteoglycans. Saphire et al (8) have reported that this interaction is mediated by cyclophilin A, i.e., a cellular protein which becomes incorporated into the viral membrane during HIV production, whereas Moulard et al. (9) have shown that the interaction is mediated by the basic residues in gp120.

The HB-19 pseudopeptide is a potent inhibitor of various X4- and R5-trop HIV-1 isolates in CD4⁺ cell lines as well as in, primary T-lymphocyte and macrophage cultures (1,10,11). HB-19 has no significant effect on the HIV-1 pseudotypes expressing glycoproteins of either Moloney murine leukemia virus or vesicular stomatitis virus, thus indicating that it is specific to the virus infection initiated by the HIV envelope glycoproteins (1, 3). The mechanism of the anti-HIV action of HB-19 is mediated through its capacity to bind cells specifically and inhibit attachment of virus particles to CD4⁺ or CD4⁻ cells (1,12, 13). At concentrations that inhibit attachment of HIV particles, HB-19 binds cells specifically and forms an irreversible complex with a cell-surface-expressed 95 kDa protein that is identified as nucleolin (3, 4, 12). Recombinant preparations of HIV-1 external envelope glycoprotein (gp120) bind partially purified preparations of nucleolin with a high affinity, comparable to that observed for the binding of gp120 to soluble CD4. Such binding is inhibited either by HB-19 or monoclonal antibodies against the V3 loop in gp120, thus suggesting that the interaction of HIV with nucleolin might occur through interactions implicating the V3 loop (3).

Nucleolin is an RNA- and protein-binding protein that has been characterized in the literature mainly as a nucleolar protein (14,15). However, several reports have demonstrated that nucleolin is also expressed on the cell surface (16-20) where it functions as a surface receptor for different ligands including the anti-HIV pseudopeptide HB-19 (3, 4, 21, 22). Studies using electron and confocal laser immunofluorescence microscopy, have confirmed that nucleolin is expressed at the cell surface where it exists in close association with the intracellular actin cytoskeleton. Cell surface expression of nucleolin is highly increased a few hours following stimulation of cell proliferation, due to induction of nucleolin mRNA and protein synthesis. Interestingly, incubation of cells with a monoclonal antibody specific to nucleolin leads to the clustering of nucleolin at the external side of the plasma membrane as revealed by electron microscopy (20). Moreover, the anti-nucleolin antibody becomes internalized at 37° C. (20) consistent with other reports that surface nucleolin can mediate intracellular import of specific ligands (21, 22). The mechanism by which nucleolin is expressed on the cell surface remains still to be elucidated. It should be noted however that nucleolin is tightly associated with the cell surface but it is readily solubilized by the non-ionic detergent Triton-X-100 (20). Three main structural domains have been determined in nucleolin: (1) the amino terminal domain containing several long stretches of acidic residues, (2) the central globular domain containing four RNA binding domains (RBDs), and (3) the extreme C-terminal domain containing nine repeats of the tripeptide motif arginine-glycine-glycine (RGG domain) (14, 15, 23).

Irreversible association of HIV particles on the surface of target cells, referred to here as anchorage, requires at least the implication of heparan sulfate proteoglycans, surface-expressed nucleolin, the CD4 receptor, and one of the members of the chemokine receptor family. Interestingly, in spite of the attachment of HIV to cells of different species not expressing CD4, anchorage of virus particles does not occur in CD4⁻ cells. Anchorage of virus particles on CD4⁺ cells can be prevented in the presence of neutralizing anti-V3 loop or anti-CD4 antibodies or treatment of cells with HB-19. The invention provides a new generation of the HB-19 pseudopeptide 5[Kψ(CH₂N)PR]TASP (10) (referred to here as HB19A) which like HB-19 presents pentavalently the Kψ(CH₂N)PR moiety, but coupled to a modified TASP template. HB-19A has anti-HIV properties identical to HB-19. Consistent with previous results obtained with HB-19 (4), HB-19A binds the cell-surface expressed nucleolin independent of the expression of cell-surface heparan and chondroitin-sulfate proteoglycans. Furthermore, cross-linking of surface bound HB-19A results in capping of surface nucleolin and its colocalization with the pseudopeptide. By using deletion constructs of nucleolin, the C-terminal tail of nucleolin containing the nine repeats of the RGG motif was identified as the HB-19A binding site. Moreover, this domain in nucleolin inhibited HIV-1 infection in a dose dependent manner by preventing attachment of virus particles to cells. The invention confirms that nucleolin is the target of the anti HIV pseudopeptide HB-19A and points out that the RGG domain is a model for the development of novel inhibitors of HIV infection.

### SUMMARY OF THE INVENTION

The invention provides for peptides involved in the attachment of microorganisms or protein ligands to the membrane of a cell. The invention also provides for therapeutic compositions comprising these peptides and methods of preventing or treating infection using these peptides. Cell-surface-expressed nucleolin binds to different protein ligands, e.g. factor J (22) or urokinase (21). The multivalent pseudopeptide HB-19 that binds the cell-surface-expressed nucleolin is a potent inhibitor of human immunodeficiency virus (HIV) infection by blocking virus particle attachment, and thus anchorage, on permissive cells. Cross-linking of surface bound HB-19A (like HB-19 but with a modified template) results in aggregation of HB-19A with surface-nucleolin, but not CD45. Consistent with its specific action, HB-19A binding to different types of cells reaches saturation at concentrations that have been reported to result in inhibition of HIV anchorage and infection. The use of Chinese hamster ovary cells (CHO cells) mutant cell lines, confirms that the binding of HB-19A to surface-nucleolin is independent of heparan and chondroitin sulfate proteoglycans. In vitro generated full-length nucleolin binds HB-19A, whereas the N-terminal part containing the acidic amino acid stretches of nucleolin does not. The use of various deletion constructs of the C-terminal part of nucleolin permitted the identification of the extreme C-terminal end of nucleolin, containing repeats of the amino acid motif RGG, as the domain that binds HB-19A. Finally, a synthetic peptide corresponding to the last C-terminal 63 amino acids inhibits HIV infection at the stage of HIV attachment to cells, showing that this domain is functional in the HIV anchorage process. The invention provides these peptides, which are involved in the attachment of microorganisms or protein ligands to the cell membrane. Additionally, the invention provides for therapeutic compositions of these peptides and methods of treatment using them.

### BRIEF DESCRIPTION OF THE DRAWINGS

FIG. 1 depicts cross-linking of surface bound HB-19A or HIV particles, which results in capping of surface nucleolin. Panels A, B, and C of FIG. 1 depict MT4 cells incubated with the biotinylated HB-19A at 20° C. for 30 min before further incubation (20° C. for 60 min) in the presence of rabbit anti-biotin antibodies (panels HB-19A-TR) to induce patching of surface bound HB-19A. After partial fixation, the co-aggregation of HB-19A with nucleolin (A and B) and CD45 (C) was investigated using murine mAbs against nucleolin and CD45. In panel B, the experiment was as in panel A but in the absence of HB-19A. Panel D of FIG. 1 depicts MT4 cells incubated (37° C., 30 min) with HIV-1 LAI before washing and further incubation (20° C. for 60 min) in the presence of anti-HIV human serum to induce aggregation of virus particles bound to cells. After partial fixation, the co-aggregation of HIV with nucleolin was investigated by using the anti-nucleolin mAb. Bound rabbit antibodies were revealed by donkey Texas Red dye (TR) conjugated anti-rabbit antibodies, human antibodies were revealed by goat TR-conjugated anti-human antibodies, while murine mAbs were revealed by goat flourescein isothiocyanate (FITC)-conjugated anti-mouse IgG. A cross-section for each staining is shown with the merge of the two colors and the respective phase contrast. Experimental conditions are described in Example 1: Experimental Procedures.

FIG. 2 depicts the interaction of HIV particles with surface nucleolin. HIV particles bound on the surface on MT4 cells were cross-linked with an anti-HIV antibody and incubated under different conditions as described in Example 1: Experimental Procedures. Panel A of FIG. 2 depicts nucleolin revealed by using anti-nucleolin antibodies and secondary antibodies coupled to gold beads of 10 nm. Typical results are presented. Each time an HIV particle was observed in the close vicinity of the plasma membrane, nucleolin signal was also detected. Note the presence of nucleolin at the external side of the plasma membrane where fiber tracts between the HIV particle and plasma membrane are observed. Panel B of FIG. 2 depicts nucleolin revealed as above with anti-HIV antibodies revealed by secondary antibodies coupled to gold beads to 15 nm. Four 10 nm beads corresponding to the nucleolin signal surround the 15 nm bead corresponding to the gp120 signal. As nucleolin is not detectable in concentrated HIV preparations, the 10 nm gold beads in Panel B correspond to surface-nucleolin, which look somehow detached from the cell surface, most probably occurring during the preparation of the sample.

FIG. 3 depicts that binding of HB-19A to cells does not require heparan- and chondroitin-sulfate proteoglycans. Panel A shows specific and non-specific binding of HB-19A to cells after incubation of HeLa P4 cells with different concentrations of the ¹²⁵I-labeled HB-19A. The specific binding was measured after washing cells in 300 nM NaCl. Panel B shows specific binding of the biotinylated HB-19A to wild type CHO K1 cells and mutant CHO 618 cells not expressing heparan- and chondroitin-sulfate proteoglycans. The experimental conditions were as described in Example 1: Experimental Procedures.

FIG. 4 depicts that binding of HB-19A to the cell-surface-expressed nucleolin does not require heparan- and chondroitin-sulfate proteoglycans. Wild type CHO K1 cells and mutant CHO cell line 677 (not expressing heparan-sulfate proteoglycans) and 618 (expressing neither heparan-nor chondroitin-sulfate proteoglycans) were incubated with the biotinylated HB-19A for the recovery of surface expressed nucleolin (Example 1: Experimental Procedures). Samples of crude nucleus-free extracts (lanes E) and surface nucleolin (lanes S) were analyzed by immunoblotting for the detection of nucleolin. Material extracted from 1.5×10⁶ and 10⁷ cells were analyzed in lanes E and S, respectively.

FIG. 5 depicts the structure of human nucleolin and deletion constructs. Panel A shows a schematic structure of nucleolin and the constructs corresponding to the N- and C-terminal parts of nucleolin. In the nucleolin structure, the positions of the long stretches of acidic domains (A1, A2, A3, A4), the bi-partite nuclear localization signal (nis), the four RNA binding domains I, II, III, IV, and the C-terminal tail containing the nine repeats of RGG are as indicated. The N-terminal part of nucleolin (referred to as NucN) and the C-terminal part of nucleolin (referred to as NucC) corresponded to amino acids 1 to 275, and 276 to 707, respectively. Panel B shows deletion constructs of the C-terminal part of nucleolin (amino acids 308-707). All of these constructs were generated as a fusion protein with GST; GST being at the N-terminal end of the fusion protein. The C-terminal part of nucleolin was referred to as R1234G for the RNA binding domains (RBDs) I, II, III, and IV, and the RGG domain at the C-terminal tail. Eight deletion constructs of R1234G were generated expressing different RBDs with or without the RGG domain: R1234, R12, R123, R234, R234G, R34, R34G, G. The − and + signs next to each construct indicates binding capacity of HB-19A.

FIG. 6 depicts HB-19A binding to the C-terminal part of nucleolin. The [³⁵S]-Met/Cys-labeled full length nucleolin, N-terminal and the C-terminal parts containing amino acids 1 to 707,1 to 275, and 276-707, respectively, were generated using an in vitro transcription-translation system. Crude labeled products were then incubated with 0, 0.5, or 1 μM of biotinylated HB-19A and the complexes formed between nucleolin and HB-19A were recovered by avidin-agarose (lanes 0, 0.5, 1). The purified proteins were eluted by heating in the electrophoresis sample buffer containing SDS, analyzed by SDS-PAGE, and the labeled bands revealed by fluorography (Example 1: Experimental Procedures). An aliquot of the crude labeled products was diluted in an equal volume of 2×-electrophoresis sample buffer containing SDS and analyzed by SDS-PAGE (lanes E). N, N/Nt, and N/Nc indicate the position of the full-length nucleolin, nucleolin/N-terminal part and nucleolin/C-terminal part, respectively. The numbers on the left indicate the position of molecular mass (in kDa) protein markers.

FIG. 7 depicts HB-19A binding to the RGG domain in the C-terminal part of nucleolin. Panel A shows expression of the GST fusion/deletion constructs of the C-terminal part of nucleolin. Aliquots of the crude bacterial extracts (containing equivalent amounts of protein) were diluted in an equal volume of 2× electrophoresis sample buffer containing SDS and analyzed by immunoblotting using anti-GST antibodies. In the different constructs, the bands lower than the most upper band represent degradation products. Partial cleavage of nucleolin has been shown to occur under different experimental conditions (3,51). Panel B shows binding of the constructs to HB-19A. Aliquots of the crude bacterial extracts (containing equivalent amounts of protein) were incubated with 5 μM of the biotinylated HB-19A and the complexes formed between a given construct and HB-19A were recovered by avidin-agarose. The purified proteins were eluted by heating in the electrophoresis sample buffer containing SDS and analyzed by immunoblotting using anti-GST antibodies. Experimental details are described in Example 1: Experimental Procedures. The numbers on the left (in panels A and B) indicate the position of molecular mass (in kDa) protein markers.

FIG. 8 depicts the dose dependent binding of HB-19A to the C-terminal part of nucleolin. Aliquots of the crude bacterial extracts (containing equivalent amounts of protein) were incubated with the biotinylated HB-19A at 0, 0.5, 1, 2, 5, and 10 μM concentrations and the complexes formed between a given construct and HB-19A were recovered by avidin-agarose. The purified proteins were eluted by heating in the electrophoresis sample buffer containing SDS and analyzed by immunoblotting using anti-GST antibodies. A section of each gel in the region of the corresponding band is presented.

FIG. 9 depicts the RGG domain of nucleolin inhibiting HIV infection by blocking attachment of virus particles to cells. Panel A shows inhibition of HIV infection by the C-terminal part of nucleolin. HIV-1 LAI infection was monitored in HeLa P4 cells by the expression of the lacZ gene (corresponding to β-galactosidase) under the control of HIV-1 LTR (1). Cells were infected in the presence of either azidothymidine (AZT) (5 μM), HB-19A (1 μM), R1234G and R1234 constructs (each at 25 μg/ml), and NP63 peptide (20 μM). The β-galactosidase activity was measured at 48 h post-infection (OD 570 nm). Each point represents the mean of duplicate samples. Panel B shows that the NP-63 peptide corresponding to the RGG domain of nucleolin inhibits HIV-1 LAI infection in a dose-dependent manner. HeLa P4 cells were infected in the presence of HP-19A (1 μM) or 2, 5, 10 and 20 μM of the NP63 peptide. The β-galactosidase activity was measured as in section A. The mean±S.D. of triplicate samples is shown. Panel C shows that the NP63 peptide inhibits HIV-1 Ba-L infection in HeLa P4-C5 cells in a dose-dependent manner. HeLa P4-C5 cells were infected in the presence of AZT (5 μM) or 1, 2.5, 5, and 10 μM of the NP63 peptide. The β-galactosidase activity was measured as in section A. The mean±S.D. of triplicate samples is shown. Note that at 10 μM of the NP63 peptide the inhibition is as efficient as AZT (which gives the background value in HeLa P4-C5 cells). Panel D shows that the NP-63 peptide corresponding to the RGG domain of nucleolin inhibits HIV attachment. Assay of HIV-1 LAI attachment (Example 1: Experimental Procedures) was performed in the presence of HB-19A (1 μM) or 2, 5, 10, and 20 μM of the NP63 peptide. The concentration of the HIV-1 core protein p24 was measured in cell extracts as an estimation of the amount of HIV attached to cells. The mean±S.D. of triplicate samples is shown.

FIG. 10 depicts the amino acid, genomic DNA and cDNA sequences of nucleolin. The sequence indicated in bold in 1 OK is the sequence of NP63.

FIG. 11 depicts the nucleotide sequence and the amino acid sequence of NP63.

### DETAILED DESCRIPTION OF THE PREFERRED EMBODIMENTS

Previously, it was reported that HIV particles can prevent the binding of HB-19 to cells and complex formation with surface nucleolin, thus suggesting that HB-19 and HIV interact with a common site in nucleolin (4). Accordingly, cross-linking of either cell-bound HB-19A or HIV particles leads to capping of surface nucleolin in accordance with ligand dependent clustering of a common surface receptor. These observations and the presence of surface nucleolin in the vicinity of HIV particles observed in electron micrographs are consistent with the implication of surface nucleolin in the HIV attachment and anchorage process. The in vitro transcription-translation system, shows that the C-terminal part of nucleolin is the domain that binds HB-19A, whereas the N-terminal part does not bind at all. This lack of interaction between the cationic HB-19A and the N-terminal part of nucleolin containing long stretches of acidic amino acid residues, and its specific binding to cells independent of expression of anionic proteoglycans, indicates that the binding of HB-19A with nucleolin is not simply a matter of charge. The specific nature of HB-19 interaction with nucleolin has also been recently demonstrated in vivo in rats. Indeed, HB-19 was shown to be preferentially taken up in vivo by lymphoid organs where it forms a stable complex with nucleolin. Thus, the molecular target of HB-19 in vivo is nucleolin (33).

Several reports have demonstrated that surface nucleolin functions as a receptor for different ligands (3,4,15, 18,19, 21, 22). Studies using electron and confocal laser immunofluorescence microscopy, confirm that nucleolin is expressed at the cell surface where it exists in close association with the intracellular actin cytoskeleton (20). As the amino acid sequence of nucleolin does not predict an hydrophobic domain to account for its anchorage into the plasma membrane, association of nucleolin with actin could be mediated by an integral membrane protein that binds both nucleolin and actin filaments. Whatever is the case, surface nucleolin is tightly associated with the plasma membrane since extensive washing of cells with high concentrations of EDTA, EGTA or NaCl has no effect (20). However, surface nucleolin is readily solubilized by treatment of cells with a non-ionic detergent. Interestingly, surface nucleolin becomes detergent-resistant following HIV anchorage to cells and is recovered along detergent insoluble membrane microdomains containing lipid raft components, such as CD59 and CD90. At the cell surface, cross-linking of HIV particles results in co-aggregation of HIV particles with CD4, CXCR4, CD56, and CD90, in addition to surface nucleolin. The aggregation of these antigens is a specific event because the surface distribution and organization of CD45 is not affected. Therefore, surface nucleolin becomes recruited in lipid rafts during HIV anchorage to cells.

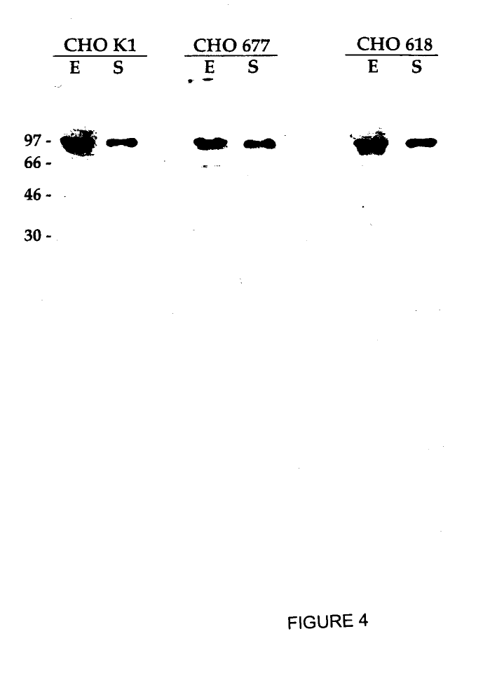

Using truncated deletion constructs of the C-terminal part of nucleolin, the C-terminal tail of nucleolin containing the RGG domain as the site that binds the pseudopeptide HB-19A was identified. Preliminary observations show that the nine RGG repeats at the C-terminal tail of nucleolin are necessary for HB-19A binding. Indeed, synthetic peptides containing four or five RGG repeats were found not to bind HB-19A and moreover not affect HIV infection. Consequently, the nine RGG repeats in the nucleolin tail are required to generate a conformation that is optimum for HB-19A binding and inhibition of HIV infection. The arginine residues in the RGG domain of nucleolin purified from eucaryotic cells are found to exist as N^{G},N^{G}-dimethylarginine (39,40). The significance of this post-translational modification of the RGG domain on the binding to HB-19A is not known. However, it is unlikely that it is essential for HB-19A binding since the nucleolin C-terminal constructs expressing the RGG domain that were generated in E. coli were shown to bind efficiently HB-19A. Previously, the RGG domain in nucleolin has been reported to bind RNA (41), rDNA (42), and subset of ribosomal proteins (43). Studies using a combination of circular dichroism and infrared spectroscopy has provided evidence that repeated β-turns are a major structural component of the RGG domain and might play a role in the formation of protein-protein interaction (41). It should also be noted that the RGG domain contains five phenylalanine residues that potentially could establish cation-π interactions (44) with the arginine and lysine residues accessible in HB-19A, or in the V3 loop of HIV. Indeed, a large amount of evidence has now established the importance of the cation-π interactions as a force for molecular recognition in a number of biological binding sites for cations. The cation-π interaction is a general noncovalent binding force, in which the face of an aromatic ring (Phe, Tyr, and Trp) provides a region of negative electrostatic potential that can bind cations with considerable strength (44,45). The cation-π interactions have been considered in such diverse systems as acetylcholine receptors, K⁺ channels, the cyclase methylation reactions involving S-adenosylmethionine, and finally in specific drug-receptor interactions (44,46,47). Recently, cation-π interactions between amino acid side chains on one hand of basic amino residues and on the other hand of an aromatic amino acid, have been shown to play an important role in intermolecular recognition at the protein-protein interface (48). In accordance with this, the RGG domain has been implicated in the process of self-annealing of nucleolin (49). Interestingly, an analogous RGG domain in the C-terminal of the heterogeneous nuclear ribonucleoprotein Al has also been shown to mediate protein-protein interactions (50). As the RGG domain in nucleolin is the binding site of HB-19A, then the C-terminal tail of nucleolin should be well exposed on the cell-surface since HB-19 and HB-19A analogues bind readily to the cell-surface-expressed nucleolin (4) (FIG. 4). The invention provides that the HIV-1 external envelope glycoprotein gp120 binds with a high affinity a partially purified preparation of nucleolin (3). The fact that the binding of gp120 to nucleolin is inhibited by HB-19 suggests that the RGG domain in surface nucleolin could represent a potential site for binding of HIV particles.

The demonstration that the anti-HIV pseudopeptide binds the C-terminal RGG domain of nucleolin further shows that nucleolin is a specific target for the action of inhibitors of HIV infection (1,4). This, and the fact that the synthetic NP63 peptide corresponding to the RGG domain inhibits HIV infection by preventing virus binding to cells, is consistent with the implication of nucleolin at an early phase of HIV infection. As the NP63 peptide inhibits HIV attachment in a dose-dependent manner, it provides a novel inhibitor of HIV infection that blocks virus particle attachment to cells. The NP63 peptide is a model for the development of novel inhibitors of HIV infection with a distinct mode of antiviral action.

For purposes of the invention, a “peptide” is a molecule comprised of a linear array of amino acid residues connected to each other in the linear array by peptide bonds. Such linear array may optionally be cyclic, i.e., the ends of the linear peptide or the side chains of amino acids within the peptide may be joined, e.g., by a chemical bond. Such peptides according to the invention may include from about three to about 500 amino acids, and may further include secondary, tertiary or quaternary structures, as well as intermolecular associations with other peptides or other non-peptide molecules. Such intermolecular associations may be through, without limitation, covalent bonding (e.g., through disulfide linkages), or through chelation, electrostatic interactions, hydrophobic interactions, hydrogen bonding, ion-dipole interactions, dipole-dipole interactions, or any combination of the above. Long polymers of amino acids linked by peptide bonds are referred to as “polypeptides.”

As used in the present specification “purified” means that the peptides are essentially free of association with other proteins or polypeptides, for example, as a purification product of recombinant host cell culture or as a purified product from a non-recombinant source.

As used in the present specification, “RGG domain of a cell-surface-expressed protein” means a peptide derived from said protein and naturally comprising RGG repeats or one of the biologically active derivatives of said peptide.

“Fragment of a RGG domain” of a cell-surface-expressed protein means a fragment of the above defined peptide comprising at least one RGG motif and preferably at least two RGG motifs. This fragment is at least 10 amino acids long and preferably 15 amino acids long.

“Biologically active derivatives” of the peptide are also part of the invention and refer to function-conservative variants, homologous proteins and peptidomimetics, as well as a hormone, an antibody or a synthetic compound, (i.e. either a peptide or non peptide molecule) that preferably retain the binding specificity and/or physiological activity of the parent peptide, as defined below. They preferably show an ability to bind to HB-19 pseudopeptide. Such binding activity may be readily determined by binding assays, e.g. by competition assay where a biologically active derivative binds to HB-19 and then prevents the binding of HB-19 to cell-surface-expressed nucleolin.

“Function-conservative variants” are those in which a given amino acid residue in a protein has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine and lysine are hydrophilic-basic amino acids and may be interchangeable. Similarly, isoleucine, a hydrophobic amino acid, may be replaced with leucine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

“Allelic variants” are more particularly encompassed, as described in greater details below.

The preferred peptide according to the invention comprises an amino acid of SEQ ID NO: 1.

In addition, certain preferred peptides according to the invention comprise, consist essentially of, or consist of an allelic variant of one RGG domain of a cell-surface-expressed protein involved in the attachment of a microorganism to the membrane of said cell. More specifically, certain preferred peptide according to the invention comprise, consist essentially of, or consist of an allelic variant of a peptide shown in SEQ ID NO: 1. As used herein, an “allelic variant” is a peptide having amino acid substitutions from a parent peptide, but retaining the binding specificity and/or physiological activity of the parent peptide. As used herein, when relating to cell-surface-expressed nucleolin, “retaining the binding specificity of the parent peptide” means being able to bind to a monoclonal or polyclonal antibody that binds to the peptide shown in SEQ ID NO: 1 with an affinity that is at least one-tenth, more preferably at least one-half, and most preferably at least as great as that of one of the peptide shown in SEQ ID NO: 1. Determination of such affinity is preferably conducted under standard competitive binding immunoassay conditions.

Peptides according to the invention can be conveniently synthesized using art recognized techniques (see e.g., Merrifield, J. Am. Chem. Soc. 85: 2149-2154).

The invention also includes preferred peptidomimetics retaining the binding specificity and/or physiological activity of the parent peptide, as described above. As used herein, a “peptidomimetic” is an organic molecule that mimics some properties of peptides, preferably their binding specificity and/or physiological activity. Preferred peptidomimetics are obtained by structural modification of peptides according to the invention, preferably using unnatural amino acids, D amino acid instead of L amino acid, conformational restraints, isoteric replacement, cyclization, or other modifications. Other preferred modifications include without limitation, those in which one or more amide bond is replaced by a non-amide bond, and/or one or more amino acid side chain is replaced by a different chemical moiety, or one of more of the N-terminus, the C-terminus or one or more side chain is protected by a protecting group, and/or double bonds and/or cyclization and/or stereospecificity is introduced into the amino acid chain to increase rigidity and/or binding affinity.

Still other preferred modifications include those intended to enhance resistance to enzymatic degradation, improvement in the bioavailability in particular by nervous, intestinal, placental and gonad tissues and more generally in the pharmacokinetic properties and especially comprise:

protecting the NH₂ and COOH hydrophilic groups by esterification (COOH) with lipophilic alcohols or by amidation (COOH) and/or by acetylation (NH₂) or added carboxyalkyl or aromatic hydrophobic chain at the NH₂ terminus;

retroinversion or reduction isomers of the CO—NH amide bonds or methylation (or ketomethylene, methyleneoxy, hydroxyethylene) of the amide functions;

substitution of L amino acids for D amino acids;

dimerisation of amino acid peptide chain.

All of these variations are well known in the art. Thus, given the peptide sequences disclosed herein, those skilled in the art are enabled to design and produce peptidomimetics having binding characteristics similar to or superior to such peptides (see e.g., Horwell et al., Bioorg. Med. Chem. 4: 1573 (1996); Liskamp et al., Recl. Trav. Chim. Pays-Bas 1: 113 (1994); Gante et al., Angew. Chem. Int. Ed. Engl. 33: 1699 (1994); Seebach et al., Helv. Chim. Acta 79: 913 (1996)).

The peptides of the present invention may be prepared in a conventional manner by peptide synthesis in liquid or solid phase by successive couplings of the different amino acid residues to be incorporated (from the N-terminal end to the C-terminal end in liquid phase, or from the C-terminal end to the N-terminal end in solid phase) wherein the N-terminal ends and the reactive side chains are previously blocked by conventional groups.

For solid phase synthesis the technique described by Merrifield may be used in particular. Alternatively, the technique described by Houbenweyl in 1974 may also be used.

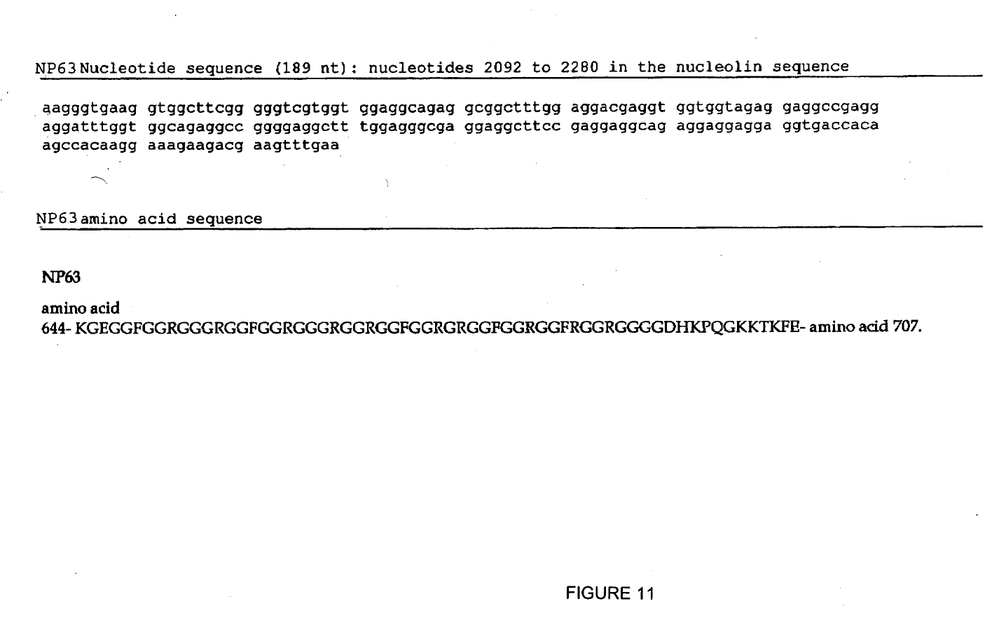

The peptides according to the present invention may also be obtained using genetic engineering methods. The nucleic acid sequence of the cDNA encoding the complete nucleolin protein appears in the PCT Patent Application No. WO 98/40480 (Hovanessian et al.) The nucleotide sequence, as well as the amino acid sequence of NP63, appear in FIG. 11 of the present application and correspond to SEQ ID NO: 2 and SEQ ID NO: 1, respectively. For the biologically active peptide derivatives of cell-surface-expressed nucleolin, a person skilled in the art will refer to the general literature to determine which appropriate codons may be used to synthesize the desired peptide.

The biologically active derivative of the peptide may be a protein, a peptide, an antibody or a synthetic compound which is either a peptide or a non peptidic molecule, such as any compound that can be synthesized by the conventional methods of organic chemistry.

Selection of the biologically active derivatives of the peptide of the invention may be performed in assessing the binding of a candidate ligand molecule to HB-19 pseudopeptide.

The invention provides for therapeutic compositions of these peptides and/or derivatives of these peptides.

The invention also provides for methods of treating or preventing infection using these peptides and/or derivatives of these peptides.

The invention also provides for polynuclotides coding for the peptides, inlcuding derivatives, of the invention. In a particular embodiment, the polynucleotide is the polynucleotide of SEQ ID NO: 2 or is a nucleotide hybridizing with the polynucleotide of SEQ ID NO: 2 under stringent conditions. As used in the present specification, stringent conditions are 0.5 mM sodium phosphate, pH 6.8; 1 mM EDTA; 1% (w/v) bovine serum albumin; 1% SDS (sodium dodecyl sulfate); incubation at 55-60° C. for 30 min.

In addition, the invention provides for NP63 or NP63 analogues as antigens to raise antibodies that bind to the C-terminal tail of surface nucleolin and block HIV infection.

### EXAMPLE 1

Experimental Procedures

Materials. The monoclonal antibody (mAb) D3 specific for human nucleolin was provided by Dr. J. S. Deng (24). Rabbit antiserum raised against a purified preparation of hamster nucleolin was provided by Dr. M. Erard. MAb CBT4 reacting with the gp120 binding site in human CD4 (5) was provided by Dr. Eugene Bosmans (clone CB-T4-2). MAb N11/20 directed against the V3 loop of HIV-1 LAI isolate was provided by Dr. J. C. Mazie. MAb 110-4 against HIV-1 LAI V3 loop was the generous gift of the Genetic Systems. MAb HB10 AB2A against CD45 was provided by Dr. R. Siraganian. HIV-1 neutralizing serum 1 and serum 2, and control human IgG were obtained through the AIDS Research and Reference Reagent Program, AIDS Program, NIAID, NIH form Dr. L. Vujcic. Rabbit anti-biotin concentrate (IgG fraction) was obtained from Enzo Dioagnostics, Inc. NY. FITC-conjugated goat anti-mouse IgG was purchased from Sigma. FITC-conjugated F(ab′)₂ fragment rabbit anti-human Ig and Texas Red dye-conjugated donkey anti-rabbit IgG were from Jackson ImmunoResearch Laboratories, Inc. PA. Texas Red dye-conjugated goat anti-human IgG was from Vector Laboratories, CA. Goat anti-rabbit antibodies coupled to gold beads of 10 nm in diameter and goat anti-human antibodies coupled to gold beads of 15 nm in diameter were obtained from Amersham Life Sciences.

Peptide Constructs. In these experiments a new generation of the HB-19 pseudopeptide 5[Kψ(CH₂N)PR]-TASP (10) was used in which the template is made of four lysine residues linked by their ε-NH₂ groups with an alanine residue introduced as a spacer at the C terminus (1). The five Kψ(CH₂N)PR moieties were then assembled on the α-amino groups of the four lysine residues and on the ε-amino group of the N-terminus lysine residue. This anti-HIV pseudopeptide with the modified TASP template is referred to here as HB-19A. The synthesis of HB-19A was performed according to the lysine residue. This anti-HIV pseudopeptide with the modified TASP template is referred here as HB-19A. The synthesis of HB-19A was performed according to the protocol described previously (1) until the introduction of the N-terminal Lys of the Kψ(CH₂N)PR motif. The reduced amide bond between Lys and Pro was formed on the resin by reductive amination of the N-protected aminoaldehyde Boc-Lys(Boc)-CHO (2.5-fold excess, twice) in dimethylformamide containing 1% acetic acid along 1 h 10). For the synthesis of the biotinylated HB-19A, the biotin moiety was coupled after the C-terminal Ala using a Fmoc-Lys(Biotin)-OH derivative. A 6-aminohexanoic acid was introduced as a spacer between Lys(Biotin) residue and the polylysine template (1). The anti-HIV cyclic peptide TW70 specific for CXCR4 was synthesized as described (25,26). The TW70 peptide has the amino acid sequence RRWCYRKDKPYRKCR, the DK has been introduced in the sequence of this peptide in order to stabilize the β-turn in the final structure. The synthetic peptide corresponding to the last 63 amino acid residues at the C-terminal tail of human nucleolin, had the following sequence (SEQ ID NO.: 1) amino acid 644-KGEGGFGGRGGGRGGFGGRGGGRGGRGGFGGRGRGGFGGRGGFRGGRGGGGDHKPQGKKTKFE-amino acid 707. All peptides were obtained at a high purity (>95%) and their integrity was controlled by matrix-associated laser desorption ionization-time-of-flight (MALDI-TOF) analysis (27). HB-19A was iodinated (5×10³ μCi/μmol) using the Bolton-Hunter reagent (NEN Life Science Products) by a procedure as recommended by the manufacturer.

Cell lines and virus preparations. CEM (clone 13) and MT-4 T lymphocyte human cell lines were propagated in RPMI-1640 (Bio-Whittaker, Verviers, Belgium). Human HeLa cells were cultured in Dulbecco's modified Eagle's medium (Gibco). Human HeLa-CD4-LTR-LacZ expressing or not expressing CCR5 were referred to as HeLa P4-C5 and HeLa P4, respectively. These HeLa cells (provided by Drs. P. Charneau and 0. Schwartz; Institut Pasteur, Paris) were cultured in Dulbecco's modified Eagle's, medium (Gibco) supplemented with G418 sulfate (500 μg/ml) for the HeLa P4 cells and with G418 sulfate (500 μg/ml)/hygromycin B (300 μg/ml) (Calbiochem-Novabiochem Corp., La Jolla, Calif.) for the HeLa P4-C5 cells (1). Chinese hamster ovary cell lines were obtained from American Type Culture Collection (ATCC): wild type cells (CHO K1) and mutant cells defective in heparan sulfate proteoglycan expression (CHO 677) or heparan and chondroitin sulfate proteoglycan expression (CHO 618) (28, 29). CHO cell lines were cultured in Ham's F12K medium. All cells were cultured with 10% (v/v) heat inactivated (56° C., 30 min) fetal calf serum (FCS; Boehringer Mannheim GmbH, Germany) and 50 IU/ml Penicillin-Streptomycin (Gibco, BRL). The HIV-1 LAI isolate was propagated and purified as described previously (1,13). The HIV-1 Ba-L isolate (30) was provided by the AIDS Program, NAID, National Institutes of Health. The MOI of the HIV-1 for different infections was 1. For the assay of HIV-1 LAI attachment, anchorage and colocalization studies purified virus was used at MOI 3.

Assay of HIV infection in HeLa CD4+cells. HIV-1 LAI infection was monitored indirectly in HeLa-CD4-LTR-/ac Z cells (HeLa P4 cells) containing the bacterial lac Z gene under the control of HIV-1 LTR. HIV-1 entry and replication result in the activation of the HIV-1 LTR leading to the expression of p-galactosidase. At 48 h post-infection, cells were lysed and assayed for β-galactosidase activity using the chlorophenol-red-β-D-galactopyranoside as a substrate. The optical density was measured at 570 nm (1). The HIV-1 Ba-L infection was monitored in HeLa-CD4-LTR-/ac Z cells but expressing also CCR5 (HeLa F4-C5 cells) as above (1).

Assay of HIV particle attachment and entry. The attachment assay was carried out at room temperature (20° C.) for 1 h in order to block viral entry (31) and potential HIV endocytosis (32). Cells were then washed extensively with culture medium containing 10% FCS to eliminate unbound HIV particles, and the amount of p24 associated with cells was measured in nucleus-free cell extracts as an estimate for the amount of HIV attached to cells by p24 Core Profile enzyme-linked immunosorbent assay (DuPont) (5, 31). In order to demonstrate that most of the p24 associated with cells represented HIV particles bound on the surface of cells, samples of cells incubated with virus were washed with PBS (containing 1 mM EDTA) before treatment with trypsin to eliminate virus bound on the cell surface as described before (1). Evidence that the virus attachment was mediated by the HIV envelope glycoprotein gp120 was provided by the capacity of anti-V3 loop mAbs N11/20 or 110/4 to inhibit the attachment process (5).

Confocal microscopy. For the colocalization experiments, MT4 cells in RPMI medium containing 10% FCS were incubated in the absence or presence of HIV (MOI 3) and the biotin-coupled HB-19A (1 μg/M) for 30 min, before washing with RPMI medium containing 1% FCS. Cells were then further incubated for 60 min in the presence of either anti-HIV serum (1/50) or rabbit anti-biotin serum (1/100) in order to cross-link virus particles and HB-19A adsorbed on the cell surface, respectively. Cells were firstly washed in RPMI/1% FCS and secondly with PBS before fixation with 0.25% paraformaldehyde (PFA). Such partially fixed cells were incubated (20° C., 45 min) with mAb D3 specific to nucleolin or to mAb HB10 specific to CD45 (10 μg/ml). After washing, cells were fixed with 3.7% PFA, washed again, and the primary antibodies were revealed by the addition of either goat Texas Red dye (TR)-conjugated anti-human antibodies, donkey TR-conjugated anti-rabbit antibodies, or goat FITC-conjugated anti-mouse IgG. Finally, cells were added in 8-well glass slides (LAB-TEK Brand, Nalge Nunc International, Naperville, Ill. USA) which were precoated with poly-L-lysine at 30 pg/ml (Sigma) and left for 15 min before washing the attached cells with PBS and proceeding for laser scanning confocal immunofluorescence microscopy (Leica TCS4D) (20).

Anchorage of HIV particles on target cells. HeLa and HeLa P4 cells were plated 24 h before the experiment in 8-well glass slides. Cells were then incubated in fresh culture medium in the absence or presence of different reagents for 30 min at 37° C. before addition of HIV-1 and further incubation but at room temperature for 1 h. Cell monolayers were then washed with PBS to eliminate unbound HIV particles before incubation in medium containing 1% FCS and the anti-gp120 mAb 110-4 (20 μg/ml) for 1 h at room temperature, in order to reveal HIV gp120 on the surface of HIV particles still remaining accessible after virus attachment. Cells were then fixed with 3.7% PFA before addition of goat FITC-labeled anti-mouse antibodies and processed for confocal microscopy (13). Under these experimental conditions the staining was observed only on the cell surface.

Electron microscopy. MT4 cells in RPMI at 10% FCS were incubated (30 min at 37° C.) with HIV-1 LAI at MOI 3. Cells were then washed with RPMI at 1% FCS and incubated (60 min, 20° C.), with anti-HIV-1 human serum (1/50) to cross-link HIV particles bound on the surface of cells. After washing in culture medium, cells were washed in PBS and partially fixed with 0.25% PFA for 10 min at 20° C. After an extensive wash in PBS, cells were incubated (45 min, 20° C.) with the biotin-conjugated mAb D3 against nucleolin (10 μl/ml). Cells washed in PBS were then fixed with 3.5% PFA before addition of rabbit anti-biotin antibodies (1:100) and incubation for 45 min at 20° C. These rabbit antibodies were revealed by goat anti-rabbit antibodies (IgG) coupled to gold beads of 10 nm in diameter, whereas human antibodies were revealed by goat anti-human antibodies (IgG) coupled to gold particles of 15 nm in diameter (1/25). Finally, cells were washed in PBS and fixed (overnight, 4° C.) in 1.6% glutaraldehyde. After further washing in PBS, cells were post-fixed with osmium tetroxyde, dehydrated in ethanol and embedded in Epon. Sections were collected on formvar-carbon-coated grids, stained with uranyl acetate and lead citrate, and observed using an electron microscope (Joel 1200EX).

Assay of HB-19A binding to cells. Cells were plated at 10⁴ cells/well in 96-well-plates for the ¹²⁵1-labeled HB-19A binding or at 10⁵ cells/well, in 24-well-plates for the biotinylated HB-19A binding. Twenty four hours later, binding experiments were performed after incubation of the cell monolayers in fresh culture medium for 1 h at room temperature (about 21° C.) in order to cool cells. It should be noted that intracellular entry of HB-19A is inhibited more than 90% at room temperature. Cells were incubated (30 min at room temperature) with different concentrations of the ¹²⁵I-labeled HB-19A or the biotinylated HB-19A before washing cells in culture medium containing 10% FCS. For total amount of binding (specific and non-specific), cells were washed 7 times with culture medium. For specific binding measurements, cells were first washed 3 times in culture medium supplemented with 150 mM NaCl thus bringing the final concentration of NaCl to 300 mM followed by 4 washings in culture medium. Washed cells were processed to reveal either the ¹²⁵I-labeled HB-19A or the biotinylated HB-19A. For the ¹²⁵1-labeled HB-19A binding, cell monolayers were extracted in 1% SDS and the radioactivity was measured in an automatic gamma counter (LKB Wallac Clini Gamma 1272). For the biotinylated HB-19A, cells were first incubated (30 min, 6° C.) in medium containing streptavidin-horseradish peroxidase conjugate (Amersham Life Science) before washing twice with medium followed by two washes in PBS containing bovine serum albumin (1%). Cell monolayers were extracted in 400 μl of lysis buffer E (20 mM Tris HCl, pH 7.6,150 mM NaCl, 5 mM MgCl₂, 0.2 mM phenylmethylsulfonyl fluoride, 5 mM p-mercaptoethanol, aprotinin (1000 U/ml) and 0.5% Triton X-100) and the nucleus-free extracts were centrifuged at 12,000 g for 10 min. Finally, ortho-phenylenediamine solution was added in the dark and the absorbance was measured at 405 nm.

Purification of cell surface associated nucleolin. These experimental conditions were previously optimized for samples from CEM and HeLa cells (3,4). Briefly, CHO cell monolayers in 150 cm² flasks (about 25×10⁶ cells/flask) were incubated in 10 ml of culture medium (Ham's F12K medium with 10% fetal calf serum) containing the biotinylated HB-19A (5 μM) for 45 min at 6° C. After washing extensively in PBS containing 1 mM EDTA (PBS-EDTA), nucleus-free cell extracts were prepared in lysis buffer E containing unlabeled HB-19A (50 μM). The complex formed between cell-surface expressed nucleolin and the biotinylated HB-19A was isolated by purification of the extracts using avidin-agarose (100 μl; ImmunoPure Immobilized Avidin from Pierce Chemical Company, USA) in PBS-EDTA. After 2 h of incubation at 4° C., the samples were washed extensively with PBS-EDTA. The purified proteins were denatured by heating in the electrophoresis sample buffer containing SDS and analyzed by SDS-PAGE. The presence of nucleolin was then revealed by immunoblotting using rabbit polyclonal antibodies against hamster nucleolin.

Immunoblotting. Samples were separated on a 10% SDS-PAGE. After electrophoresis, proteins were transferred to a 0.22 mm polyvinylidine difluoride sheet (PVDF, Bio-Rad). The electrophoretic blots were saturated with casein-based blocking buffer (Genosys), washed extensively before incubation with rabbit polyclonal antibodies against hamster nucleolin. After extensive washing, the filter was treated with horseradish peroxidase (HRP)-conjugated rabbit anti-mouse immunoglobulin (Amersham Pharmacia Biotech). The reacting bands were visualized with an enhanced chemiluminescence (ECL) reagent and by exposure to autoradiography film (Amersham Pharmacia Biotech).

Generation of deletion constructs of human nucleolin. The pcDNA4 Nuc, pcDNA4 NucN and pcDNA4 NucC plasmids encode the full-length human nucleolin ORF, its N-terminal part (corresponding to the first 275 amino-acids) and its C-terminal part (corresponding to the last 433 amino-acids), respectively. The full-length human nucleolin and its truncated derivatives were generated by PCR using high-fidelity DNA polymerase (Expand High Fidelity PCR System, Roche) and the following oligonucleotides: NucN-F/NucC-R for Nuc, NucN-F/NucN-R for NucN and NucC-F/NucC-R for NucC.

NucN-F:5′-GGGGATCCATGGTGAAGCTCGCGAAGGCAGG-3′
NucN-R; 5′-CCGAATTCTTCTTTGACAGGCTCTTCCTCCT-3′
NucC-F:5′-GGGGATCCGAAG CACCTGGAAAACGAAAG-3′
NucC-R:5′-GGGAATTCCTATTCAAACTTCGTC TTCTTTCCTTG-3′

The PCR products, containing BamHI and EcoRI restriction sites (underlined), were digested by the corresponding restriction enzymes and inserted between BamHI and EcoRI sites of the pcDNA4His/Max C plasmid (Invitrogen).

Deletion constructs of the C-terminal half of human nucleolin were generated by PCR using human nucleolin cDNA as template. R1234G (1234 corresponding to the four RNA Binding Domains and G to the Gly/Arg rich RGG domain) encodes the full-length C-terminal part of nucleolin and was amplified with R1 N and RGGC primers. The other constructs encode the same part, deleted from one or more domains: R1234 (R1N/-R4C), R12 (RIN/R2C), R123 (R1 N/R3C), R234 (R2N/R4C), R234G (R2N/RGGC), R34 (R3N/R4C), R34G (R3N/RGGC) and RGG (RGGN/RGGC), using the primer pairs indicated in parenthesis. The sequence of oligonucleotides were as follow.

RIN:5′-GGATCCAATCTCTTTGTTGGAAACCTAAAC-3′,
R2N:5′-GGATCCACACTTTTGGCTAAAAATCTCCCT-3′,
R2C:5′-GAATTCTTTTGATTCACCACTCCAAGTGCT-3′,
R3N:5′-GGATCCACTCTGGTTTTAAGCAACCTCTCC-3′,
R3C:5′-GAATTCTTTGGATGGCTGGCTTCTGGCATT-3′,
R4N:5′-GGATCCACTCTGTTTGTCAAAGGCCTGTCT-3′
R4C:5′-GAATTCAGGTTTGGCCCAGTCCAAGGTAAC-3′,
RGGN:5′-GGATCCAAGGGTGAAGGTGGCTTCGGGGGT-3′,
RGGC:5′-GAATTCCTATTCAAACTTCGTCTTCTTTCC-3′.

PCR products were sub-cloned in pCR2.1 TOPO plasmid (Invitrogen) by T/A cloning, removed using BamHI and a EcoRI sites inserted at their 5′- and 3′-ends, respectively (underlined) and cloned between the corresponding sites of pGEX-N3 plasmid (Pharmacia Biotech), in frame to glutathione S-transferase (GST).

In vitro transcription and translation of human nucleolin. The TNT coupled reticulocyte lysate system (Promega) was used to produce the wild-type nucleolin (pcDNA4 Nuc) and the N- and C-terminal parts of nucleolin (pcDNA4 NucN and pcDNA4 NucC containing amino acids 1-308 and 309-707) by in vitro transcription/translation. Reactions were performed by using 2 μg of each plasmid and 20 μCi of specified by the manufacturer.

To assay the HB-19A binding capacity of the generated nucleolin and nucleolin truncated constructs, 25 μl of translation products were diluted in 25 μl of modified BI buffer (20 mM Tris-HCl, pH 7.6,100 mM NaCl, 50 mM KCl, 2 mM EDTA, 2% Triton X-100,1000 U/ml aprotinin), centrifuged at 12,000 g for 10 min at 4° C. and diluted in 150 μl of PBS. Binding reactions were performed by incubating 200 μl of each diluted lysates with the biotinylated HB-19A for 1 h at 4° C. Complexes formed between nucleolin deletion constructs and biotinylated HB-19A were isolated by purification of the extracts using avidin-agarose (100 μl) in PBS-EDTA (12). After 2 h of incubation at 4° C., the samples were washed extensively with PBS-EDTA. The purified proteins were eluted by heating in the electrophoresis sample buffer containing SDS and analyzed by SDS-PAGE. The [³⁵S]methionine/cysteine labeled proteins were revealed by fluorography.

Expression and purification of recombinant nucleolin constructs. Escherichia coli (E. coli) BL21 (DE3) cells were transformed with each pGEX-N3 plasmid coding for truncated derivatives of the C-terminal part of human nucleolin. Cells were grown overnight at 37° C. in 100 ml of Terrific Broth containing 100 μg of ampicillin per ml. After 1:50 dilution in 100 ml of fresh medium, cells were grown at 30° C. to a 600 nm optical density of approximately 0.5 (6 h) and induced with 0.5 mM isopropyl-1-thio-D-galactopyranoside (IPTG) for an additional 2 h. Bacteria were pelleted at 4,000 g for 10 min at 4° C., resuspended in 5 ml of PBS containing 0.2 mM phenylmethylsulphonyl flouride (PMSF) and 1 mM DTT, and frozen at −20° C. overnight. After being thawed on ice, the bacterial suspensions were frozen again in a dry ice-methanol bath, thawed on ice, sonicated three times for 30 sec each time on ice, adjusted to 1% (wt/vol) with Triton X-100, and centrifuged at 9,000 g for 20 min at 4° C. The presence of proteins in cell extracts was monitored by SDS-Page with Coomassie blue staining, and their respective concentration estimated and adjusted.

To assay HB-19A binding capacity, bacterial extracts were first diluted 5-fold in PBS-EDTA before centrifugation at 12,000 g for 10 min at 4° C. Supernatants were then incubated for 1 h at 4° C. with the biotinylated HB-19A and the complexes were recovered on avidin-agarose (100 μl) in PBS-FDTA by incubation for 2 h at 4° C. The samples were washed 3 times with PBS-EDTA and twice with PBS-EDTA containing additional 150 mM NaCl (resulting to a final concentration of 300 mM NaCl) to eliminate unbound material and also the non-specifically bound proteins. The purified proteins were eluted by heating in the electrophoresis sample buffer containing SDS and analyzed by immunoblotting using anti-GST monoclonal antibody B-14 (Santa Cruz Biotechnology, Inc.).

Purification of the GST fusion proteins. The truncated nucleolin constructs R1234G and R1234 were purified directly from bacterial lysates using glutathione sepharose 4B affinity chromatography (Amersham Pharmacia Biotech.) by a procedure as recommended by the manufacturer. The proteins were recovered in the glutathione elution buffer. The purified proteins were dialyzed against PBS and stored at −80° C. Assay of the peptides for their activity.

Assay of peptides for their activity. Each peptide is tested at different concentrations to investigate their capacity to inhibit HIV infection. Each peptide is investigated for its binding capacity to the HB-19, using biotin-coupled HB-19.

Raising polyclonal antibodies against NP63. The antigenicity of NP63 and NP63 in which the arginine residues are dimethylated is tested in this procedure. Rabbits are injected with each peptide supplemented with Freund's adjuvant. The peptides can be coupled to ovalbumin. Sera is collected before each boosting with the antigen and the titer of the antibody production is tested by ELISA. Sera are also tested by Western blot experiments to show their reactivity with nucleolin. Finally, sera is tested to investigate its capacity to neutralize HIV infection.

### EXAMPLE 2

### Several Components are Required for Anchorage of HIV Particles on the Cell-Surface

The stable (i.e., functional) association of HIV particles with the plasma membrane of target cells, referred to as “anchorage,” can be monitored by inducing the cross-linking of adsorbed virions with anti-gp120 antibodies. When particles are bound on the surface of CD4⁺ permissive cells, the antibody-induced cross-linking leads to their aggregation at one pole of the cell. This antibody-dependent cross-linking of HIV particles on CD4⁺ cells permits monitoring the anchorage of virus particles in the plasma membrane by confocal laser immunofluorescense microscopy. The fluorescent signal of HIV is found only at the cell surface, because it disappears when scanning is performed at intracellular level (13). In contrast, although HIV particles are able to attach to CD4-negative cells, cross-linking with anti-HIV antibodies washes out particles, thus confirming that attachment alone is not sufficient for anchorage of virions (not shown). Besides the requirement of CD4, the proper anchorage of HIV particles on target cells is coordinated by other surface components, such as heparan sulfate proteoglycans, nucleolin, and chemokine receptors. Consequently, anchorage is inhibited either by neutralizing anti-CD4 antibodies, the fibroblast growth factor 2 which binds heparan sulfate proteoglycans, the pseudopeptides HB-19 or HB-19A which bind nucleolin, and the TW70 peptide which binds CXCR4 ((1, 4, 13,26).

### EXAMPLE 3

HB-19A Induced Capping of Surface Nucleolin.

By using the biotinylated HB-19 it was previously shown that HB-19 binds specifically the cell-surface-expressed nucleolin and forms an irreversible complex with it (3, 4,12). The nucleolin-HB-19 complex then becomes internalized at 37° C. but not at reduced temperatures (33). At 20° C. HB-19 remains attached to the cell surface without entering the cytoplasm. Similarly, internalization of the anti-nucleolin antibody is also blocked at reduced temperatures thus suggesting that internalization via nucleolin occurs by an active process (20). For these reasons, cell binding with HB-19A was performed at 20° C.

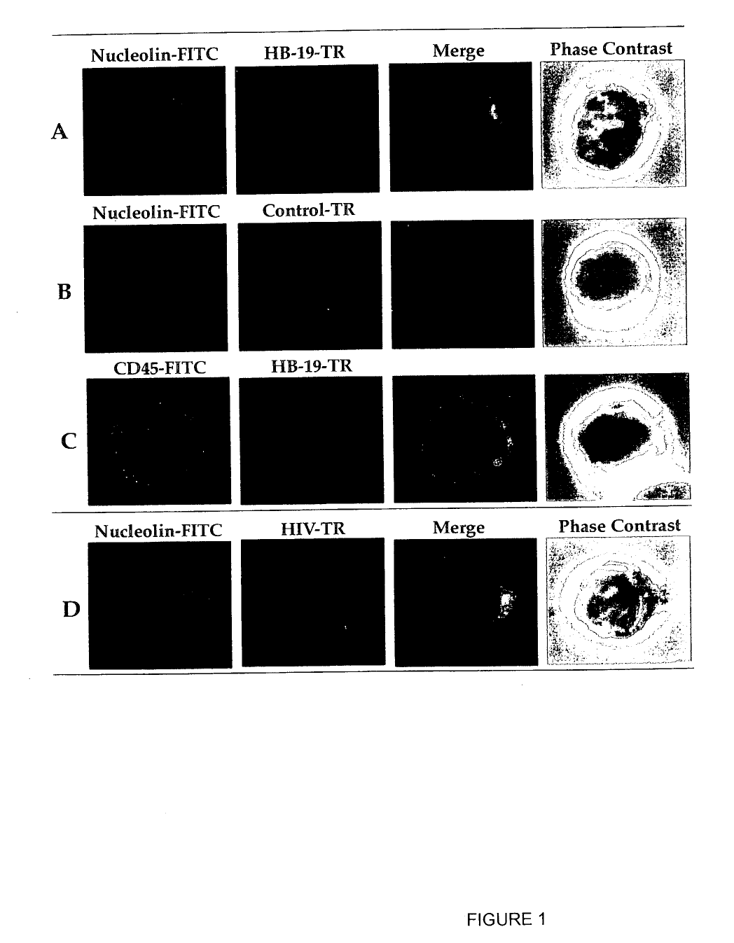

In general, the cross-linking of a ligand leads to the clustering or capping of its surface receptor. Accordingly, this Example demonstrates distribution of surface nucleolin following the cross-linking of the biotinylated HB-19A using anti-biotin antibodies. The biotinylated HB-19A binding to MT4 cells was carried out for a short period at 20° C. before washing cells and further incubation with anti-biotin antibodies to induce lateral aggregation of HB-19A. Cells were then partially fixed with 0.25% PFA before adding the monoclonal antibody against nucleolin in order to reveal the steady state distribution of nucleolin at the plasma membrane. Under such experimental conditions, the nucleolin signal was patched at one pole of the cell, which coincided with the HB-19A signal (FIG. 1A). On the other hand, in control cells incubated without HB-19A the nucleolin signal was detected as evenly distributed in the plasma membrane and in a diffused state (FIG. 1B). The ligand dependent capping of surface nucleolin observed in the presence of HB-19A was a specific event since the distribution of another surface protein CD45 did not seem to be affected much. Indeed, the CD45 signal was found to be more or less distributed evenly at the periphery of cells, and when HB-19A was aggregated the distribution of CD45 was not significantly modified. The enhancement of the CD45 signal at positions when the HB-19A spots were observed was most probably a non-specific effect due to patching of membrane components (FIG. 1C). Interestingly, capping of surface nucleolin was also observed in cells following cross-linking of HIV particles bound to cells and consequently the HIV signal became colocalized with that of nucleolin (FIG. 1D). Under such experimental conditions HIV particles also cause capping of CD4 and CXCR4, but not CD45. It should be noted that the binding of the parental HB-19 to surface nucleolin is prevented by preincubation of cells with HIV particles thus suggesting the existence of a competition between HIV and HB-19 to bind surface nucleolin (4). This, and the capacity of HB-19A and HIV to cluster surface nucleolin, are consistent with the nucleolin being a common target for both ligands.

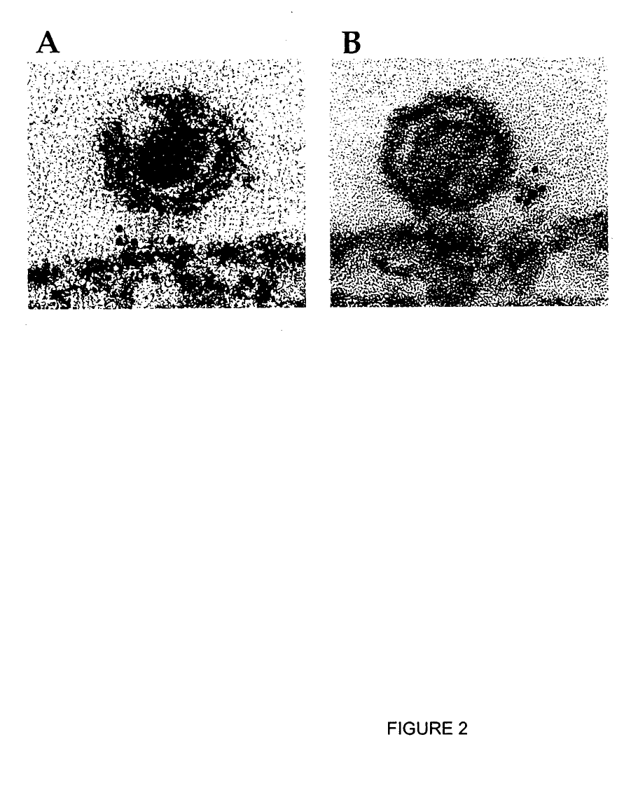

As shown by electron microscopy, when surface nucleolin is cross-linked by the anti-nucleolin antibody it becomes clustered at the external side of the plasma membrane (20). In MT4 cells with anchored HIV, surface nucleolin also becomes abundant at a region in close contact with HIV particles (FIG. 2A). This effect is revealed by the presence of several gold particles of 10 nm in diameter corresponding to the anti-nucleolin antibody. In some cases, the nucleolin signal colocalized with that of the gp120 on the HIV particle. An example is shown in FIG. 2B in which a 15 nm gold particle corresponding to the anti-gp120 antibody is surrounded by four 10 nm gold particles corresponding to the anti-nucleolin antibody. The detection of gp120 on the surface of HIV particles was dependent on its association with the plasma membrane (FIG. 2B). This is due to the fact that gp120 being associated non-covalently with gp41 becomes readily shed off the HIV particles during manipulation (34,35), particularly during washing virus particles by centrifugation. In the experimental procedure presented in this Example there are 12 steps of centrifugation of HIV infected cells before processing for electron microscopy. Moreover, the antibody against gp120 exerts an additional tension on gp120 leading to the shedding of gp120-antibody complexes from the part of HIV virions not trapped with components of the plasma membrane. The presence of nucleolin at the external side of the plasma membrane, where fiber tracts between the HIV particle and plasma membrane are observed (36), further illustrates the colocalization of HIV particles with surface nucleolin and demonstrates the existence of a direct contact of HIV particles with nucleolin.

### EXAMPLE 4

The binding of HB-19A to the Cell-Surface-Expressed Nucleolin does not Require Heparan and Chondroitin-Sulfate Proteoglycans.

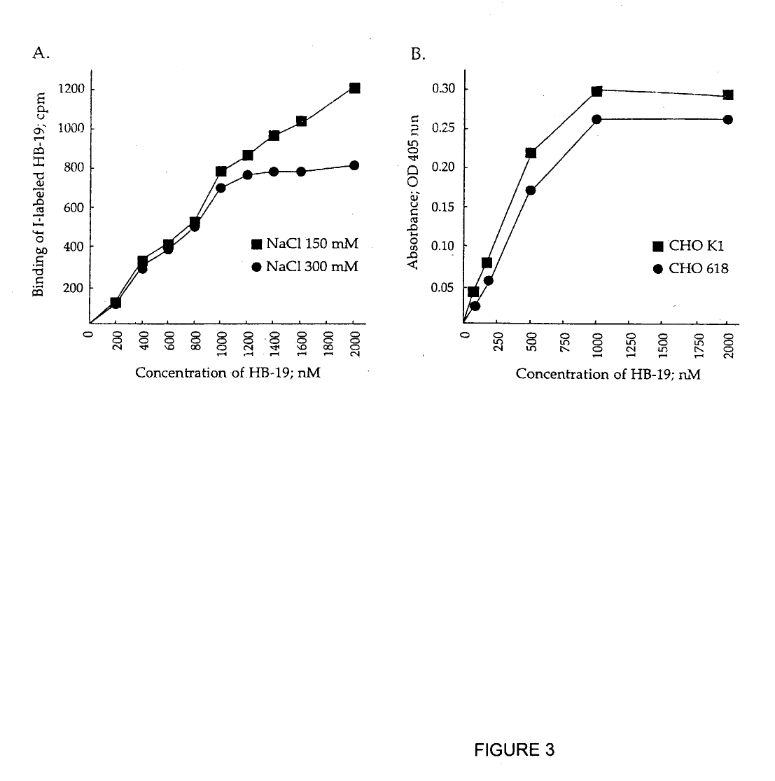

The specific and non-specific binding of HB-19A to HeLa P4 cell monolayers can be monitored by washing cells at 300 and 150 mM NaCl, respectively. In cells washed at 300 mM NaCl, specific binding occurs in a dose-dependent manner and reaches a saturation at 1 μM of HB-19A (FIG. 3A), at a dose which has been shown to inhibit more than 95% HIV infection in different types of cells (1,10). Interestingly, the non-specific binding mostly occurred when the specific binding of HB-19A had reached saturation. Indeed, at concentrations of HB-19A less than 1 μM there was no apparent difference between the binding values in the absence or presence of 300 mM NaCl wash.

In order to evaluate the potential requirement of heparan- and chondroitin-sulfate proteoglycans in the capacity of HB-19A to bind cells, we used Chinese hamster ovary mutant cell lines that are deficient in the expression of heparan sulfate proteoglycans (CHO 677) or both heparan- and chondroitin-sulfate proteoglycans (CHO 618) (28,29). The HB-19A binding profile on the wild type CHO cells (CHO K1) was similar to that observed for the HeLa cells, in that the binding was dose dependent and reached a saturation at 1 μM of HB-19A (FIG. 3B). Interestingly, no significant difference was observed in the HB-19A binding profile between the CHO K1 and CHO 618 that was devoid of heparan- and chondroitin-sulfate proteoglycans (FIG. 3B). Moreover, no significant difference was observed in the kinetics of HB-19A binding to the wild type and mutant CHO cell lines (not shown).

In order to demonstrate that the binding of HB-19A to nucleolin is independent of heparan- and chondroitin-sulfate proteoglycans, the capacity of HB-19A to form a complex with the surface nucleolin expressed in different types of CHO cell lines was monitored. For this purpose, cells were incubated with the biotinylated HB-19A at 6° C. and the complex formed between surface-nucleolin and HB-19A was recovered by purification of nucleus-free extracts using avidin-agarose. The presence of nucleolin was then revealed by immunoblotting. FIG. 4 shows the recovery of surface nucleolin at comparable levels from the different CHO cell lines independent of the expression of heparan- and chondroitin-sulfate proteoglycans. These observations are consistent with previous results showing that HB-19A binding to cells and complex formation with surface nucleolin is independent of heparan-sulfate proteoglycans (4). In the different CHO cell clones, the amount of cell-surface nucleolin was estimated to be less than 15% of the total nucleolin recovered in the nucleus-free cell extracts.

### EXAMPLE 5

The C-Terminal Tail of Nucleolin containing the RGG Repeats is the Site of Binding to HB-19A.

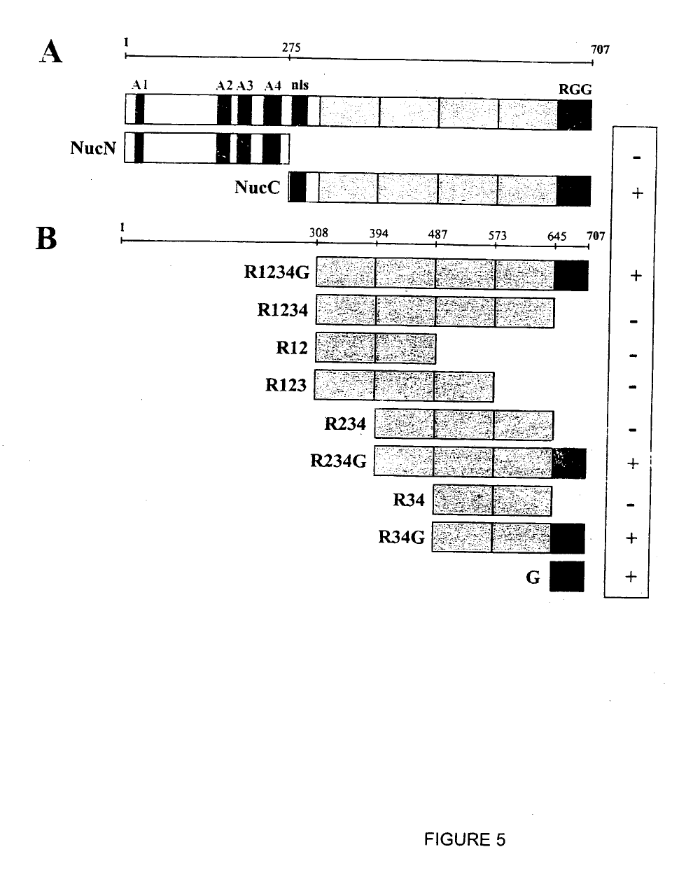

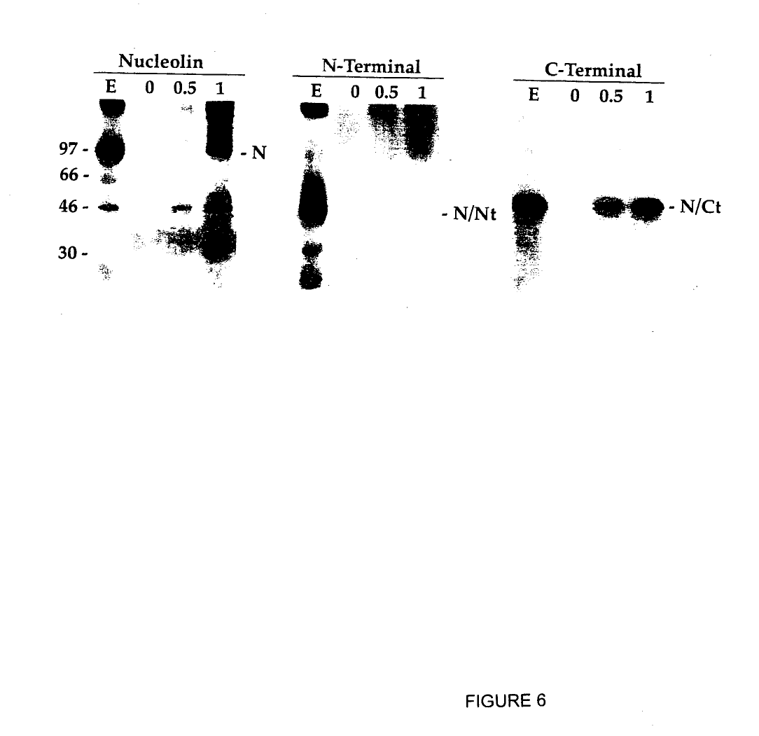

Because of the cationic nature of HB-19 analogues, it was previously proposed that the N-terminal part of nucleolin containing long stretches of acidic amino acids represents a potential binding site for these pseudopeptides (3). In order to illustrate this effect, truncated constructs of nucleolin corresponding to its N- and C-terminal parts by in vitro transcription/translation in the rabbit reticulocyte lysate system were generated, because the full length nucleolin and the N-terminal part of nucleolin cannot be expressed in E. coli (37). [³⁵S]-Met/Cys-labeled full length nucleolin, N-terminal and the C-terminal parts containing amino acids 1 to 707, 1 to 308, and 309-707, respectively, were produced in the reticulocyte lysate system (FIGS. 5A and 6). Crude labeled products were then incubated with different concentrations of the biotinylated HB-19A and the complex recovered by avidin-agarose. The full-length nucleolin was found to bind HB-19A. Intriguingly, the N-terminal part of nucleolin containing the acidic stretches did not bind at all, whereas the C-terminal part was bound efficiently to HB-19A (FIG. 6).

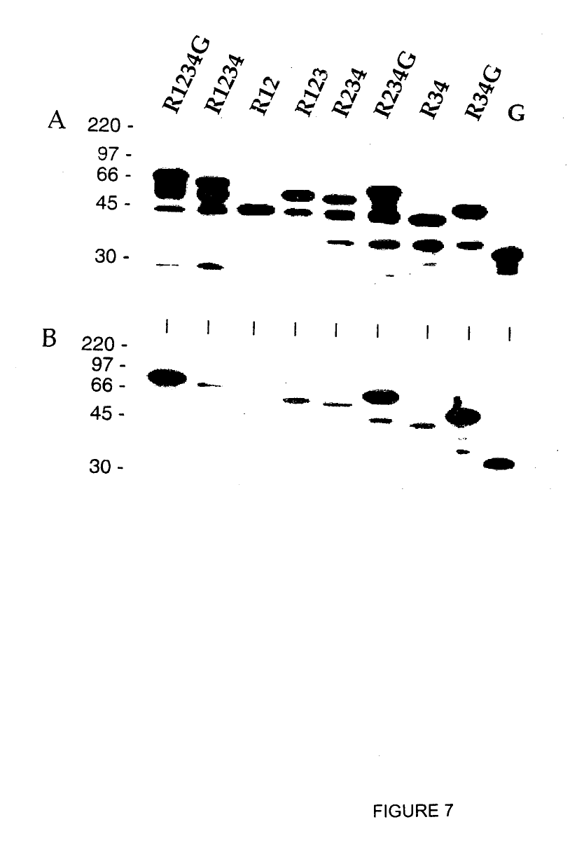

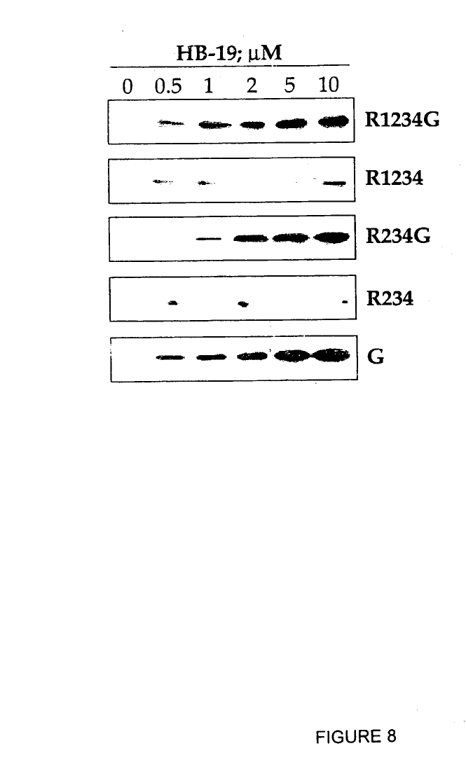

In order to further characterize the binding domain of HB-19A, truncated constructs of the C-terminal part of nucleolin in E. coli fused with GST were generated as a tag to permit their detection by antibodies against GST. The C-terminal part of nucleolin was referred to as R1234G; R1234 for the RNA binding domains (RBDs) I, II, III, and IV, and G for the RGG domain at the C-terminal tail. Eight deletion constructs of R1234G were generated expressing different RBDs with or without the RGG domain: R1234, R12, R123, R234, R234G, R34, R34G, G (FIG. 5B). The HB-19A binding capacity of each construct was then investigated by incubation of crude bacterial extracts with the biotinylated HB-19A. The results show that the presence of the RGG domain determines the HB-19A binding capacity of a given construct in the C-terminal part of nucleolin. Furthermore, the RGG domain alone is sufficient for binding (FIG. 7). The faint binding of some constructs lacking the RGG domain was most probably nonspecific because it was not dependent on the concentration of HB-19A (FIG. 8, lanes R1234, R234). On the other hand, the binding of constructs containing the RGG domain was increased with the dose of HB-19A and reached a maximum value at 5 μM of HB-19A (FIG. 8, lanes 1234G, R234G, G).

### EXAMPLE 6

The C-Terminal part of Nucleolin containing the RGG Domain Inhibits HIV Infection.

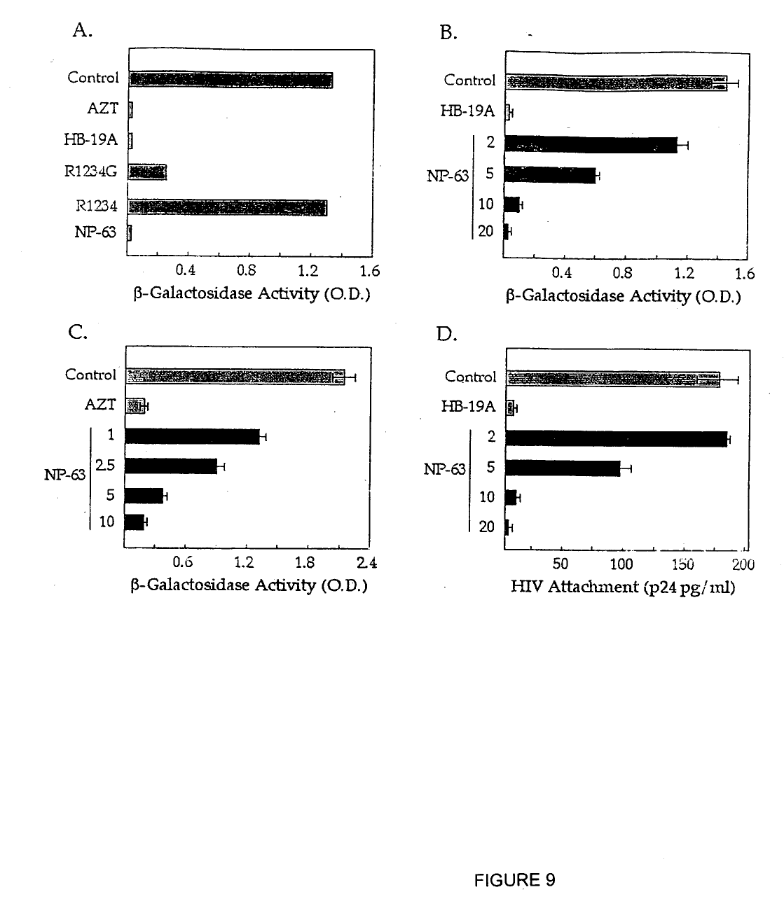

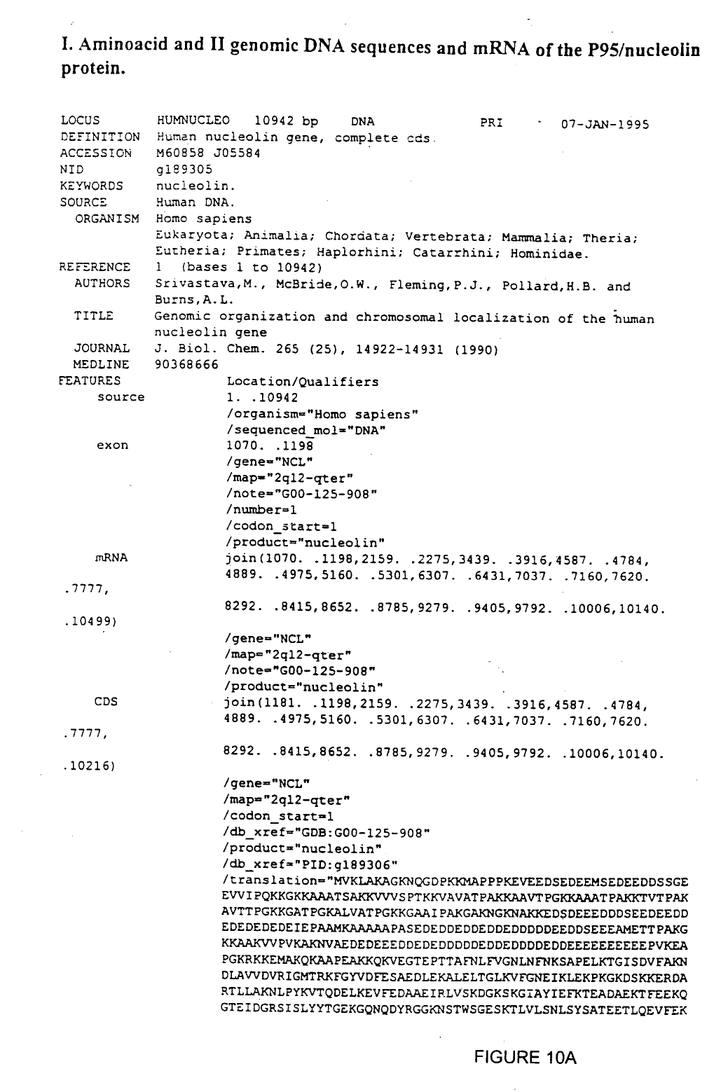

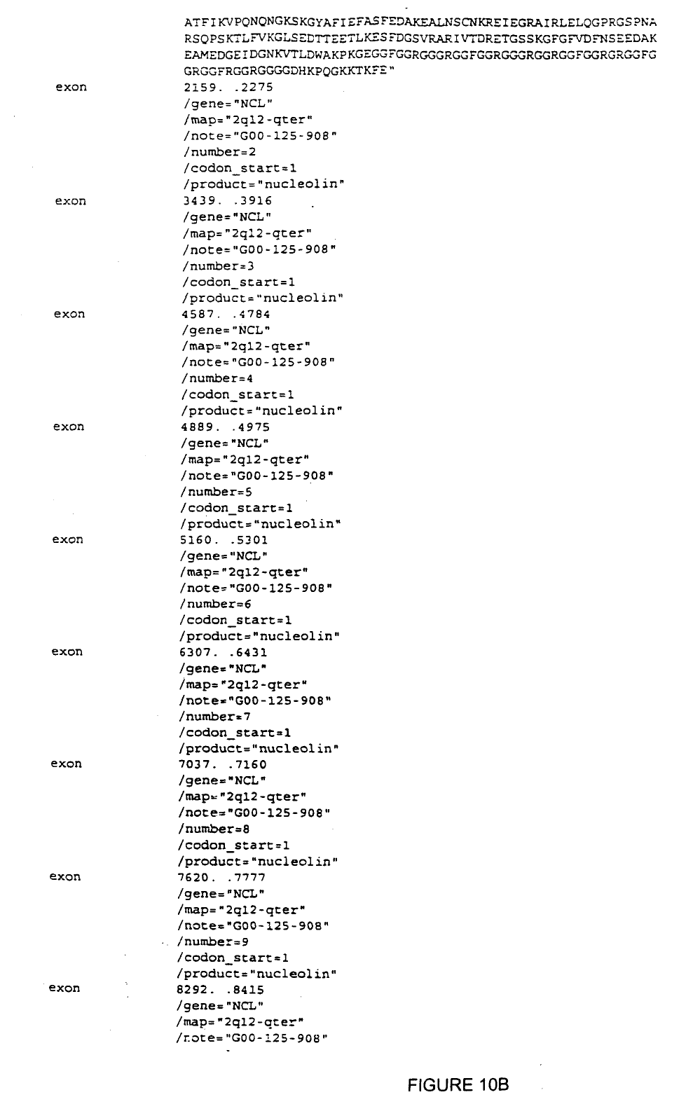

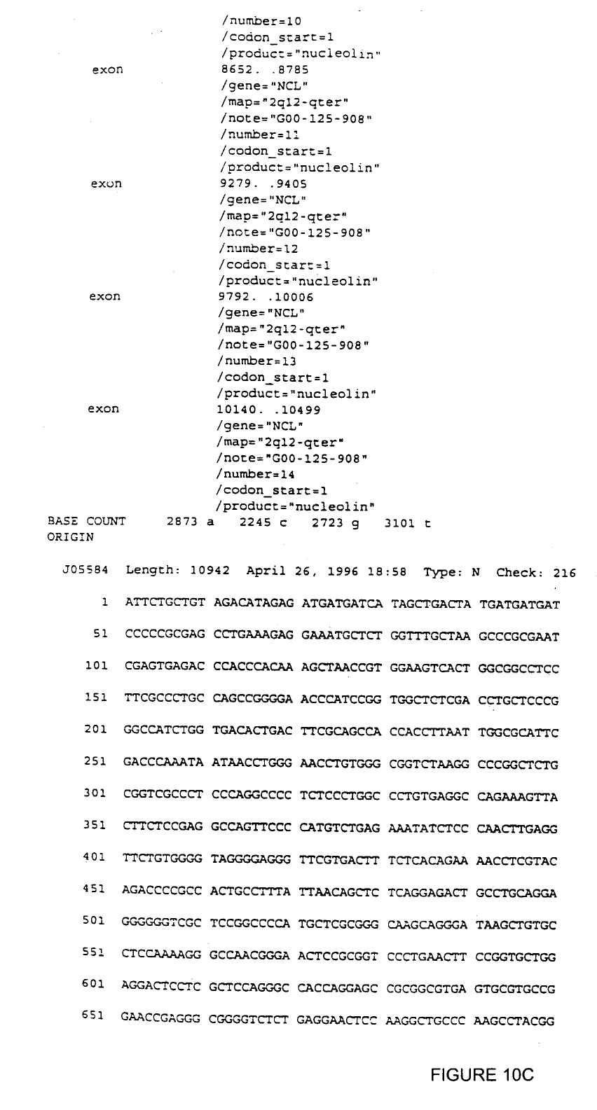

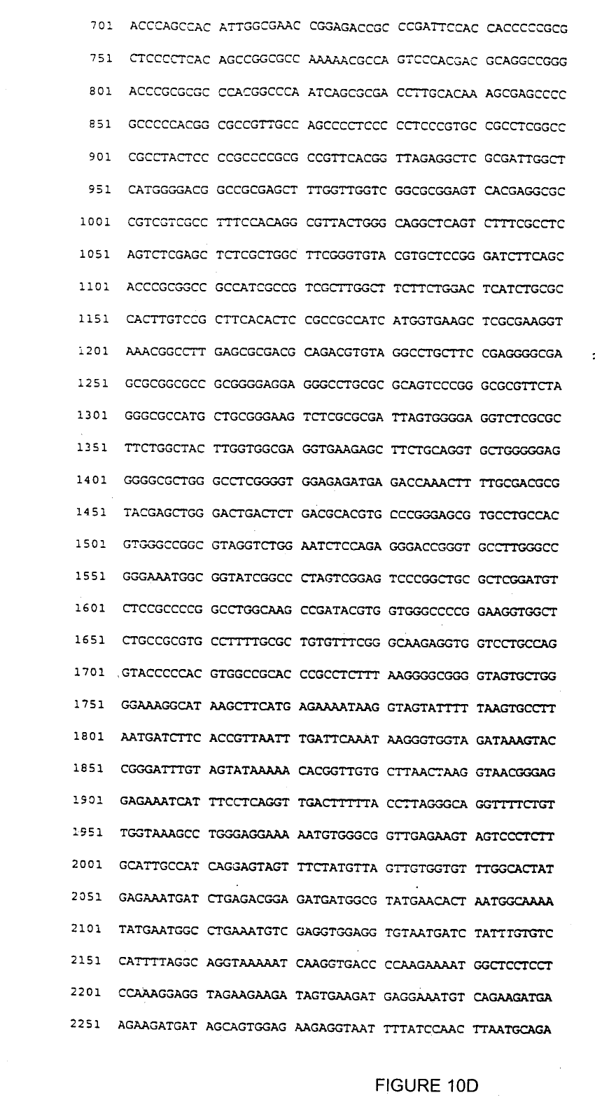

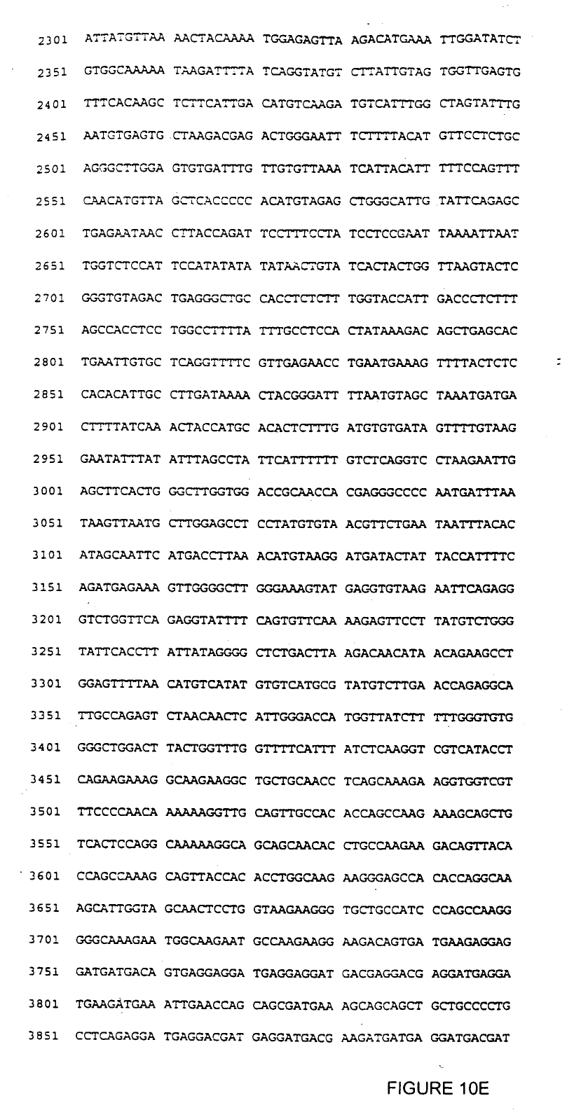

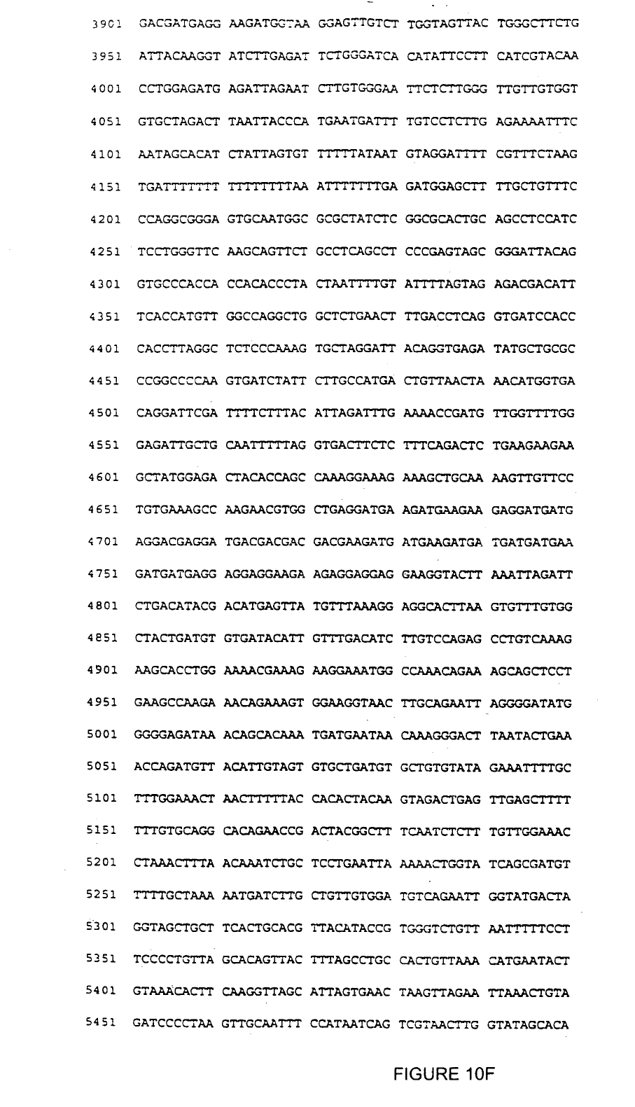

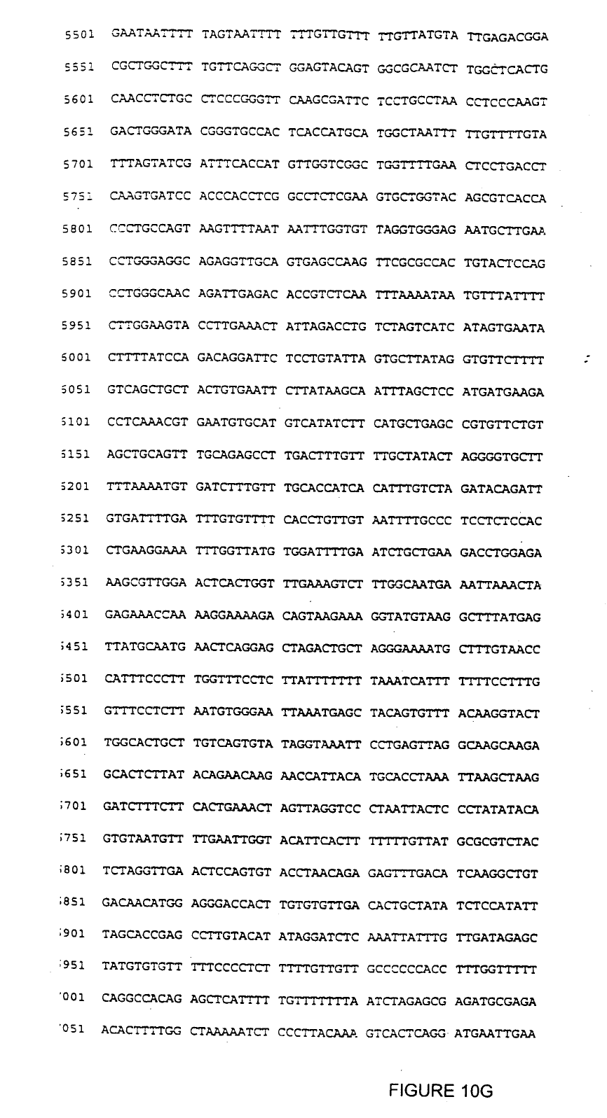

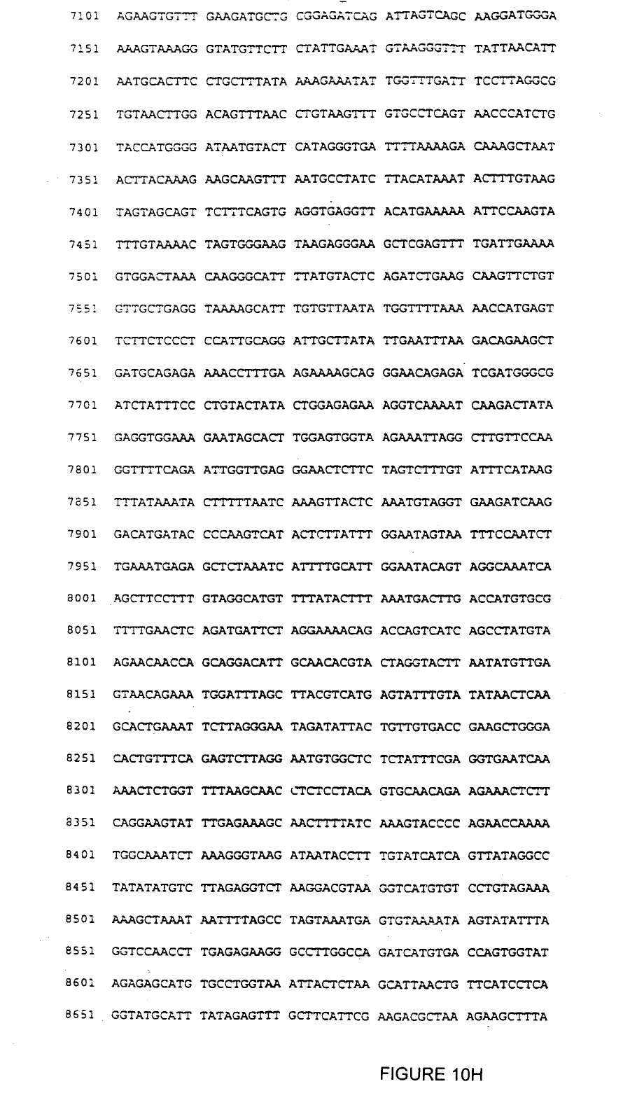

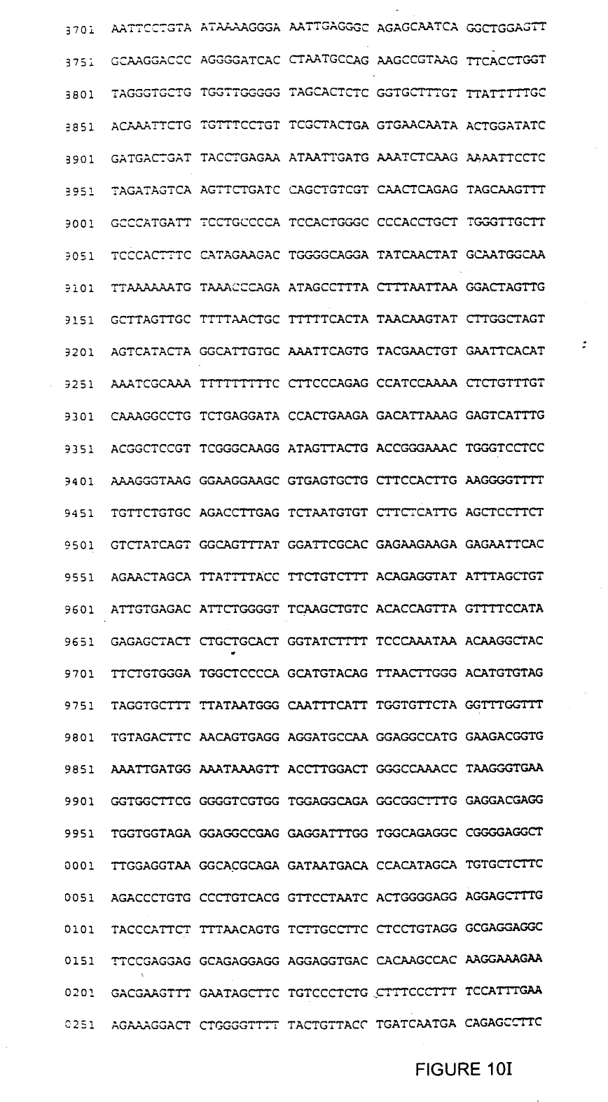

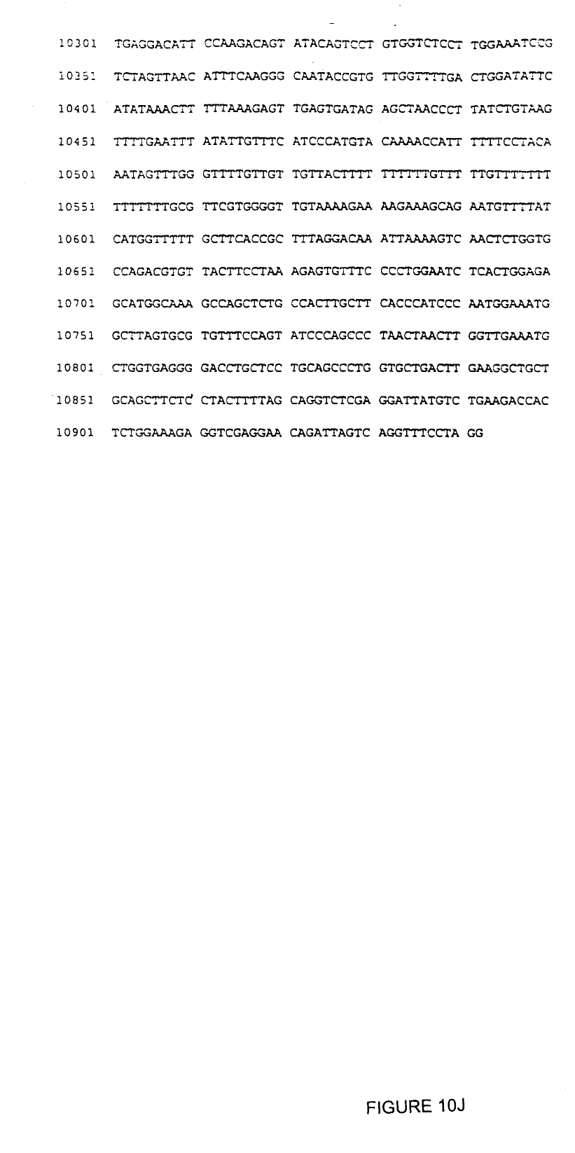

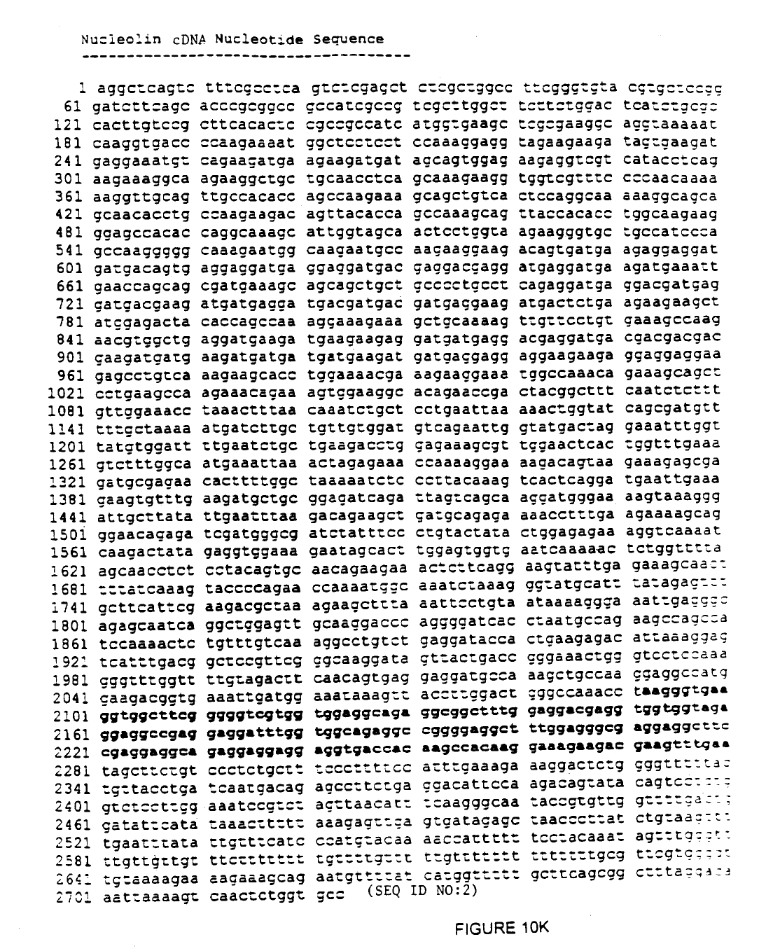

The nucleolin constructs R1234G and R1234 were purified by using a glutathione sepharose column and assayed for their potential capacity to inhibit HIV-1 infection in the HeLa P4 cells experimental model (FIG. 9). The HeLa P4 cells provide an efficient system to monitor inhibitors of the T lymphocyte-tropic HIV-1 LAI attachment and entry into cells. For example, pretreatment of cells with HB-19 or HB-19A leads to inhibition of HIV entry at the level of HIV attachment to cells (1, 4) (FIG. 9). In the presence of the R1234G construct, HIV infection was inhibited by more than 75%, whereas the R1234 construct lacking the RGG domain had no effect. In view of this result, a peptide corresponding to the last 63 amino acids of nucleolin containing the nine repeats of RGG was synthesized. This peptide, referred to as NP63, for nucleolin peptide containing 63 amino acids, inhibited completely HIV-1 LAI infection (FIG. 9A). The NP63 effect was most probably mediated via its affinity to bind HIV. Indeed, cells pretreated with NP63 manifested no resistance to HIV when the culture supernatants containing the peptide were replaced by fresh culture media before addition to HIV (not shown). In contrast, previously it was demonstrated that HB-19 pretreated cells remain resistant to HIV even after several hours after removal of the culture medium containing the inhibitor (1). NP63 inhibited HIV-1 LAI infection in a dose dependent manner with an IC₅₀ value of 5 μM (FIG. 9B). This was the consequence of inhibition of virus attachment to cells in a dose dependent manner (FIG. 9D). The IC₅₀ value for the inhibition of HIV-1 LAI attachment by NP63 was estimated to be 5 μM. The inhibitory effect of NP63 was not restricted to T lymphocyte-tropic HIV-1 isolates, because it also inhibited macrophage-tropic HIV-1 Ba-L isolate in a dose-dependent manner with an IC₅₀ value of 1 μM (FIG. 9C). Therefore, HIV-1 Ba-L appears to be more sensitive to the inhibitory effect of NP63 compared to HIV-1 LAI. Whether this latter is due to the presence of lower number of basic residues in the V3 loop of macrophage-tropic HIV-1 isolates compared to that of T lymphocyte-tropic HIV-1 isolates (38), remains to be investigated.

### EXAMPLE 7

Determination of the Minimum Domain of NP63 Capable of Inhibiting HIV Infection, and of the Significance of Individual Residues within NP63

Determination of the minimum domain of NP63

The RGG domain contains 9 repeats of RGG. It is important, therefore, to determine the minimum number of repeats necessary for the anti-HIV action. The RGG domain ends with this sequence DHKPQGKKTKFE. The following peptides can be synthesized and tested for their activity against HIV infection.

NP50:
amino acid 644-
KGEGGFGGRGGGRGGFGGRGGGRGGRGGFGGRGRGGFGGRGGFRGGRG
GG - amino acid 693
NP40:
amino acid 667-
GGRGGFGGRGRGGFGGRGGFRGGRGGGGDHKPQGKKTKFE- amino acid
707.
NP25:
amino acid 667- GRGGFGGRGRGGFGGRGGFRGGRGG - amino acid 691.
NP16:
amino acid 691- GGGGDHKPQGKKTKFE - amino acid 707.
NP15:
amino acid 667- GRGGFGGRGGFGGRG - amino acid 681.

Determination of the Significance of Dimethylation of the Arginine Residues in NP63.

The arginine residues in the RGG domain of nucleolin purified from eukaryotic cells are found to exist as N^{G},N^{G}-dimethylarginine (39, 40). It is important to determine the significance of this post-translational modification of the RGG domain on the binding to HB-19. Dimethylation of the arginine residues will generate a more rigid and a stable NP63 structure which can have a more potent anti-HIV action. Moreover, as the dimethylation of the arginine residues is the natural form of the RGG domain, neutralizing antibodies can be generated from it.

The Significance of the Phenylalanine Residues in NP63 (cation-π Interactions).

The RGG domain contains five phenylalanine residues that can establish cation-π interaction (44) with the arginine and lysine residues accessible in HB-19, or in the V3 loop of HIV. Indeed, a large amount of evidence establishes the importance of the cation-π interactions as a force for molecular recognition in a number of biological binding sites for cations. The cation-π interaction is a general noncovalent binding force, in which the face of an aromatic ring (Phe, Tyr, and Trp) provides a region of negative electrostatic potential that can bind cations with considerable strength (44, 45). The cation-π interactions have been considered in such diverse systems as acetylcholine receptors, K⁺ channels, the cyclase methylation reactions involving S-adenosylmethionine, and finally in specific drug-receptor interactions (44, 46, 47). Recently, cation-π interactions between amino acid side chains on one hand of basic amino residues, and the other hand of an aromatic amino acid, have been shown to play an important role in intermolecular recognition at the protein-protein interface (48).

For this purpose, two mutated peptides can be used to show the role of these residues:

1) NP63 in which the aromatic phenylalanine residues can be changed to non-aromatic alanine residue. This mutated peptide can illustrate the significance of the phenylalanine residue in the antiviral action of NP63.

2) NP63 in which the aromatic phenylalanine residues can be changed to another aromatic residue tryptophane and tyrosine. This mutated peptide can confirm that the anti-HIV action of NP63 is due to cation-π interactions. In addition, the mutated peptide can have a more potent anti-HIV action due to more stable structure.

The Significance of the Arginine and the Glycine Residues in NP63.

Because the RGG domain contains repeated motifs containing arginine and glycine residues, new peptides, with either the arginine or the glycine residues mutated to serine can be used to demonstrate the role of these residues.

The Synthesis of NP63 Analogues with Enhanced Activity against HIV Infection.

One or several of the arginine residues of the sequence can be advantageously replaced by positively charged residues like lysine, hydroxy-lysine, and ornithine. On the other hand, one or several pseudopeptide bonds can be introduced at certain positions in the backbone instead of the amide bond, in order to generate a more stable peptide that could resist the action of proteases. These pseudopeptide bonds can be (among others) of methylene amino, retro inverso or carba type.

### REFERENCES

1. Nisole, S., Krust, B., Dam, E., Bianco, A., Seddiki, N., Loaec, S., Callebaut, C., Guichard, G., Muller, S., Briand, J. P., and Hovanessian, A. G. (2000) AIDS Res. Hum. Retroviruses 16, 237-249.

2. Ugolini, S., Mondor, I., and Sattentau, Q. J. (1999) Trends Microbiol. 7, 144-149.

3. Callebaut, C., Blanco, J., Benkirane, N., Krust, B., Jacotot, E., Guichard, G., Seddiki, N., Svab, J., Dam, E., Muller, S., Briand, J. P., and Hovanessian, A. G. (1998) J. Biol. Chem. 273, 21988-21997.

4. Nisole, S., Krust, B., Callebaut, C., Guichard, G., Muller, S., Briand, J. P., and Hovanessian, A. G. (1999) J. Biol. Chem. 274, 27875-27884.

5. Valenzuela, A., Blanco, J., Krust, B., Franco, R., and Hovanessian, A. G. (1997) J. Virol. 71, 8289-8298.

6. Roderiquez, G., Oravecz, T., Yanagishita, M., Chequer Bou-Habib, D., Mostowski, H., and Norcross, M. A. (1995) J. Virol. 69, 2233-2239.

7. Mondor, I., Ugolini, S., and Sattentau, Q. J. (1998) J. Virol. 72(5), 3623-3634.

8. Saphire, A. C. S., Bobardt, M. D., and Gallay, P. A. (1999) The EMBO J. 18, 6771-6785.

9. Moulard, M., Lortat-Jacob, H., Mondor, I., Roca, G., Wyatt, R., Sodroski, J., Zhao, L., Olson, W., Kwong, P. D., and Sattentau, Q. J. (2000) J. Virol. 74, 1948-1960.

10. Callebaut, C., Jacotot, E., Guichard, G., Krust, B., Rey-Cuille, M. A., Cointe, D., Benkirane, N., Blanco, J., Muller, S., Briand, J. P., and Hovanessian, A. G. (1966) Virology 218, 181-192.

11. Seddiki, N., Nisole, S., Krust, B., Callebaut, C., Guichard, G., Muller, S., Briand, J. P., and Hovanessian, A. G. (1999) AIDS Res. & Hum. Retroviruses 15, 381-390.

12. Callebaut, C., Jacotot, E., Krust, B., Guichar, G., Blanco, J., Svab, J., Muller, S., Briand, J. P., and Hovanessian, A. G. (1997) J. Biol. Chem. 272, 7159-7166

13. Callebaut, C., Nisole, S., Briand, J. P., Krust, B., and Hovanessian, A. G. (2001) Virology 281, 248-264.

14. Ginisty, H., Sicard, H., Roger, B., and Bouvet, P. (1999) J. Cell Science 112, 761-772.

15. Srivastava, M., and Pollard, H. B. (1999) FASEB J. 13,1911-1922.

16. Sememkovich, C. F., Ostlund, R. E., Olson, M. O., and Yang, J. W. (1990) Biochemistry 29, 9708-9713.

17. Kleinman, H. K., Weeks, B. S., Cannon, F. B., Sweeney, T. M., Sephel, G. C., Clement, B., Zain, M., Olson, M. 0. J., Jucker, M., and Burrous, B. A. (1991)Arch. Biochem. Biophys. 290, 320-325.

18. Krantz, S., Salazar, R., Brandt, R., Kellerman, J., and Lottspeich, F. (1995) Biochim. Biophys. Acta 1266,109-112.

19. De Verdugo, U. R., Selinkgra, H. C., Huber, M., Kramer, B., Kellermann, J., Hofschneider, P. H., and Kandolf, R. (1995) J. Virol. 69, 6751-6757.

20. Hovanessian, A. G., Puvion-Dutilleul, F., Nisole, S., Svab, J., Perret, E., Deng, J. S., and Krust, B. (2000) Exp. Cell Res. 261, 312-328.

21. Dumler, I., Stepanova, V., Jerke, U., Mayboroda, O. A., Vogel, F., Bouvet, P., Thachuk, V., Haller, H., and Gulba, D.C. (1999) Current Biology 9, 1468-1476.

22. Larrucea, S., Cambronero, R., Gonzalez-Rubio, C., Fraile, B., Gamallo, C., Fontan, G., and Lopez-Trascasa, M. (1999) Biochem. Biophys. Res. Commun. 266, 51-57.

23. Srivastava, M., Fleming, P. J., Pollard, H. B., and Burns, A. L. (1989) FEBS Letters 250, 99-105.

24. Deng, J. -S., Baillou, B., and Hofineister, J. K. (1966) Mol. Biol. Rep. 23,191-195.

25. Murakami, T., Nakajima, T., Koyanagi, Y., Tachibana, K., Fujii, N., Tamamura, H., Yoshida, N., Waki, M., Matsumoto, A., Yoshie, O., Kishimoto, T., Yamamoto, N., and Nagasawa, T. (1997) J. Exp. Med. 186,1389-1393.

26. Tamamura, H., Waki, M., Imai, M., Otaka, A., Ibuka, T., Waki, K., Miyamoto, K., Matsumoto, A., Murakami, T., Nakashima, H., Yamamoto, N., and Fujii, N. (1998) Bioorg. & Med. Chemistry 6, 473-479.

27. Limal, D., Briand, J. -P., Dalbon, P., and Joliver, M. (1998) J. Peptide Res. 52,121-129.

28. Esko, J. D., Weinke, J. L., Taylor, W. H., Ekborg, G. Roden, L., Anantharamaiah, G., and Gawish, A. (1987) J. Biol. Chem. 262, 12189-12195.

29. Esko, J. D., Rostand, K. S., and Weinke, J. L. (1988) Science 241,1092-1096.

30. Gartner, S., Markovits, P., Markovitz, D. M., Kaplan, M. H., Gallo, R. C., and Popovic, M. (1986) Science 233(4760), 215-219.

31. Krust, B., Callebaut, C., and Hovanessian, A. G. (1993) AIDS Res. Hum. Retroviruses 9, 1087-1090.

32. Maréchal, V., Clavel, F., Heard, J.-M., and Schwartz, 0. (1998) J. Virol. 72, 2208-2212.

33. Krust, B., Vienet, R., Cardona, A., Rougeot, C., Jacotot, E., Callebaut, C., Guichard, G., Briand, J. P., Grognet, J. M., Hovanessian, A. G., and Edelman, L. (2001) Proc. Natl. Acad. Sci. USA 98,14090-14095.

34. Schneider, J., Kaaden, O., Copeland, T. D., Oroszlan, S., and Hunsmann, G. (1986) J. Gen. Virol. 67, 2533-2538.

35. Wyatt, R., Kwong, P. D., Desjardins, E., Sweet, R. W., Robinson, J., Hendrickson, W. A., and Sodroski, J. G. (1998) Nature 393, 705-711.

36. Stauber, R. H., Rulong, S., Palm, G., and Tarasova, N. I. (1999) Biochem. Biophys. Res. Commun. 258, 695-702.

37. Hanakahi, L. A., Dempsey, L. A., Li, M. -J., and Maizels, N. (1997) Proc. Natl. Acad. Sci. USA 94, 3605-3610.

38. De Jong, J. -J., De Ronde, A., Keulen, W., Tersmette, M., and Goudsmit, J. (1992) J. Virol. 66, 6777-6780.

39. Lischwe, M. A., Cook, R. G., Ahn, Y. S., Yeoman, L. C., and Busch, H. (1985) Amer. Chem. Soc. 24, 6025-6028.

40. Lapeyre, B., Amalric, F., Ghaffari, S. H., Venkatarama Rao, S. V., Dumbar, T. S., and Olson, M. O. J. (1986) J. Biol. Chem. 261, 9167-9173.

41. Ghisolfi, L., Joseph, G., Amalric, F., and Erard, M. (1992) J. Biol. Chem. 267, 2955-2959.

42. Hanakahi, L. A., Sun, H., and Maizels, N. (1999) J. Biol. Chem. 274,15908-15912.

43. Bouvet, P., Diaz, J. -J., Kindbeiter, K., Madjar, J. -J., and Amairic, F. (1998) J. Biol. Chem. 273,19025-19029.

44. Dougherty, D. A. (1996) Science 271, 163-168.

45. Gallivan, J. P., and Dougherty, D. A. (1999) Proc. Natl. Acad. Sci. USA 96, 9459-9464.

46. Mecozzi, S., West, A. P., and Dougherty, D. A. (1996) Proc. Natl. Acad. Scie. USA 93,10566-10571.

47. Zhong, W., Gallivan, J. P., Zhang, Y., Li, L., Lester, H. A., and Dougherty, D. A. (1998) Proc. Natl. Acad. Sci. USA 95, 12088-12093.

48. Pletneva, E. V., Laederach, A. T., Fulton, D. B., and Kostié, N. M. (2001) J. Am. Chem. Soc. 123, 6232-6245.

49. Hanakahi, L. A., Bu, Z., and Maizels, N. (2000) Biochem. Biophys. Res. Commun. 39, 15493-15499.

50. Cartegni, L. Maconi, M., Morandi, E., Cobianchi, F., Riva, S., and Biamonti, G. (1966) J. Mol. Biol. 259, 337-348.

51. Fang, S. -H., and Yeh, N. -H. (1993) Exp. Cell Res. 208, 48-53.

What is claimed is:

1. A purified peptide wherein said peptide comprises one RGG domain of a cell-surface-expressed protein, or a fragment thereof, involved in the attachment of a microorganism or a protein ligand to the membrane of said cell.

2. The purified peptide of claim 1, wherein said cell-surface-expressed protein is nucleolin.

3. The purified peptide of claim 2, wherein said peptide comprises the C-terminal part of nucleolin.

4. The purified peptide of claim 3, wherein said peptide comprises the nine RGG repeats of nucleolin.

5. The purified peptide of claim 4, wherein said peptide comprises the 63 last amino acids of the C-terminal part of said nucleolin.

6. The purified peptide of claim 1, wherein said peptide is comprises of said RGG domain of the cell-surface-protein.

7. The purified peptide of claim 4, wherein said peptide is comprised of the 63 last amino acids of the C-terminal part of said nucleolin.

8. The purified peptide of claim 5, wherein said peptide is SEQ ID NO: 1.

9. A purified polynucleotide wherein said polynucleotide codes for the peptide of claim 1.

10. A purified polynucleotide wherein said polynucleotide codes for the peptide of SEQ ID NO: 1.

11. The purified peptide of claim 1, wherein said microorganism is a virus.

12. The purified peptide of claim 11, wherein said virus is a VIH.

13. A therapeutic composition for preventing or treating a virus infection wherein said composition comprises a peptide comprising one RGG domain of a cell-surface-expressed protein, or a fragment thereof, involved in a microorganism infection, or a biologically active derivative thereof, along with a pharmaceutically acceptable carrier.

14. The therapeutic composition of claim 13, wherein said cell-surface expressed protein is nucleolin.

15. The therapeutic composition of claim 14, wherein said peptide comprises the C-terminal part of nucleolin.

16. The therapeutic composition of claim 15, wherein said peptide comprises the nine RGG repeats of nucleolin.

17. The therapeutic composition of claim 16, wherein said peptides comprises the 63 last amino acids of the C-terminal part of said nucleolin.

18. The therapeutic composition of claim 13, wherein said composition comprises said RGG domain of the cell-surface-protein or a biologically active derivative thereof.

19. The therapeutic composition of claim 18, wherein said composition comprises the 63 last amino acids of the C-terminal part of said nucleolin or a biologically active derivative thereof.

20. The therapeutic composition of claim 19, wherein said composition comprises a peptide having SEQ ID NO: 1.

21. A method for preventing or treating a microorganism infection in a mammal, wherein said method comprises administration to said mammal of a pharmaceutical composition according to claim 13.

22. The method of claim 20, wherein said microorganism is a virus.

23. The method of claim 22, wherein said virus is a HIV.

24. A purified polynucleotide wherein said polynucleotide is SEQ ID NO: 2 or hybridizes with SEQ ID NO: 2 under stringent conditions.

25. An antibody, wherein the antibody is raised against NP63 or an NP63 analogue, binds to the C-terminal tail of surface nucleolin, and blocks HIV infection.


SEQUENCE LISTING (25 sequences)

SEQ ID NO: 1 - protein, 63 aa, Homo sapiens
KGEGGFGGRGGGRGGFGGRGGGRGGRGGFGGRGRGGFGGRGGFRGGRGGGGDHKPQGKKTKFE

SEQ ID NO: 2 - DNA, 189 bp, Homo sapiens
aagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaa

SEQ ID NO: 3 - protein, 707 aa, Homo sapiens
MVKLAKAGKNQGDPKKMAPPPKEVEEDSEDEEMSEDEEDDSSGEEVVIPQKKGKKAAATSAKKVVVSPTKKVAVATPAKKAAVTPGKKAAATPAKKTVTPAKAVTTPGKKGATPGKALVATPGKKGAAIPAKGAKNGKNAKKEDSDEEEDDDSEEDEEDDEDEDEDEDEIEPAAMKAAAAAPASEDEDDEDDEDDEDDDDDEEDDSEEEAMETTPAKGKKAAKVVPVKAKNVAEDEDEEEDDEDEDDDDDEDDEDDDDEDDEEEEEEEEEEPVKEAPGKRKKEMAKQKAAPEAKKQKVEGTEPTTAFNLFVGNLNFNKSAPELKTGISDVFAKNDLAVVDVRIGMTRKFGYVDFESAEDLEKALELTGLKVFGNEIKLEKPKGKDSKKERDARTLLAKNLPYKVTQDELKEVFEDAAEIRLVSKDGKSKGIAYIEFKTEADAEKTFEEKQGTEIDGRSISLYYTGEKGQNQDYRGGKNSTWSGESKTLVLSNLSYSATEETLQEVFEKATFIKVPQNQNGKSKGYAFIEFASFEDAKEALNSCNKREIEGRAIRLELQGPRGSPNARSQPSKTLFVKGLSEDTTEETLKESFDGSVRARIVTDRETGSSKGFGFVDFNSEEDAKEAMEDGEIDGNKVTLDWAKPKGEGGFGGRGGGRGGFGGRGGGRGGRGGFGGRGRGGFGGRGGFRGGRGGGGDHKPQGKKTKFE

SEQ ID NO: 4 - DNA, 10942 bp, Homo sapiens
attctgctgtagacatagagatgatgatcatagctgactatgatgatgatcccccgcgagcctgaaagaggaaatgctctggtttgctaagcccgcgaatcgagtgagacccacccacaaagctaaccgtggaagtcactggcggcctccttcgccctgccagccggggaacccatccggtggctctcgacctgctcccgggccatctggtgacactgacttcgcagccaccaccttaattggcgcattcgacccaaataataacctgggaacctgtgggcggtctaaggcccggctctgcggtcgccctcccaggcccctctccctggccctgtgaggccagaaagttacttctccgaggccagttccccatgtctgagaaatatctcccaacttgaggttctgtggggtaggggagggttcgtgactttctcacagaaaacctcgtacagaccccgccactgcctttattaacagctctcaggagactgcctgcaggaggggggtcgctccggccccatgctcgcgggcaagcagggataagctgtgcctccaaaagggccaacgggaactccgcggtccctgaacttccggtgctggaggactcctcgctccagggccaccaggagccgcggcgtgagtgcgtgccggaaccgagggcggggtctctgaggaactccaaggctgcccaagcctacggacccagccacattggcgaaccggagaccgcccgattccaccacccccgcgctcccctcacagccggcgccaaaaacgccagtcccacgacgcaggccgggacccgcgcgcccacggcccaatcagcgcgaccttgcacaaagcgagccccgcccccacggcgccgttgccagcccctccccctcccgtgccgcctcggcccgcctactccccgccccgcgccgttcacggttagaggctcgcgattggctcatggggacggccgcgagctttggttggtcggcgcggagtcacgaggcgccgtcgtcgcctttccacaggcgttactgggcaggctcagtctttcgcctcagtctcgagctctcgctggcttcgggtgtacgtgctccgggatcttcagcacccgcggccgccatcgccgtcgcttggcttcttctggactcatctgcgccacttgtccgcttcacactccgccgccatcatggtgaagctcgcgaaggtaaacggccttgagcgcgacgcagacgtgtaggcctgcttccgaggggcgagcgcggcgccgcggggaggagggcctgcgcgcagtcccgggcgcgttctagggcgccatgctgcgggaagtctcgcgcgattagtggggaggtctcgcgcttctggctacttggtggcgaggtgaagagcttctgcaggtgctgggggagggggcgctgggcctcggggtggagagatgagaccaaacttttgcgacgcgtacgagctgggactgactctgacgcacgtgcccgggagcgtgcctgccacgtgggccggcgtaggtctggaatctccagagggaccgggtgccttgggccgggaaatggcggtatcggccctagtcggagtcccggctgcgctcggatgtctccgccccggcctggcaagccgatacgtggtgggccccggaaggtggctctgccgcgtgccttttgcgctgtgtttcgggcaagaggtggtcctgccaggtacccccacgtggccgcacccgcctctttaaggggcggggtagtgctggggaaaggcataagcttcatgagaaaataaggtagtatttttaagtgccttaatgatcttcaccgttaatttgattcaaataagggtggtagataaagtaccgggatttgtagtataaaaacacggttgtgcttaactaaggtaacgggaggagaaatcatttcctcaggttgactttttaccttagggcaggttttctgttggtaaagcctgggaggaaaaatgtgggcggttgagaagtagtccctcttgcattgccatcaggagtagtttctatgttagttgtggtgtttggcactatgagaaatgatctgagacggagatgatggcgtatgaacactaatggcaaaatatgaatggcctgaaatgtcgaggtggaggtgtaatgatctatttgtgtccattttaggcaggtaaaaatcaaggtgaccccaagaaaatggctcctcctccaaaggaggtagaagaagatagtgaagatgaggaaatgtcagaagatgaagaagatgatagcagtggagaagaggtaattttatccaacttaatgcagaattatgttaaaactacaaaatggagagttaagacatgaaattggatatctgtggcaaaaataagattttatcaggtatgtcttattgtagtggttgagtgtttcacaagctcttcattgacatgtcaagatgtcatttggctagtatttgaatgtgagtgctaagacgagactgggaatttcttttacatgttcctctgcagggcttggagtgtgatttgttgtgttaaatcattacatttttccagtttcaacatgttagctcacccccacatgtagagctgggcattgtattcagagctgagaataaccttaccagattcctttcctatcctccgaattaaaattaattggtctccattccatatatatataactgtatcactactggttaagtactcgggtgtagactgagggctgccacctctctttggtaccattgaccctctttagccacctcctggccttttatttgcctccactataaagacagctgagcactgaattgtgctcaggttttcgttgagaacctgaatgaaagttttactctccacacattgccttgataaaactacgggattttaatgtagctaaatgatgacttttatcaaactaccatgcacactctttgatgtgtgatagttttgtaaggaatatttatatttagcctattcattttttgtctcaggtcctaagaattgagcttcactgggcttggtggaccgcaaccacgagggccccaatgatttaataagttaatgcttggagcctcctatgtgtaacgttctgaataatttacacatagcaattcatgaccttaaacatgtaaggatgatactattaccattttcagatgagaaagttggggcttgggaaagtatgaggtgtaagaattcagagggtctggttcagaggtattttcagtgttcaaaagagttccttatgtctgggtattcaccttattataggggctctgacttaagacaacataacagaagcctggagttttaacatgtcatatgtgtcatgcgtatgtcttgaaccagaggcattgccagagtctaacaactcattgggaccatggttatctttttgggtgtggggctggacttactggtttggttttcatttatctcaaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatggtaaggagttgtcttggtagttactgggcttctgattacaaggtatcttgagattctgggatcacatattccttcatcgtacaacctggagatgagattagaatcttgtgggaattctcttgggttgttgtggtgtgctagacttaattacccatgaatgattttgtcctcttgagaaaatttcaatagcacatctattagtgttttttataatgtaggattttcgtttctaagtgatttttttttttttttaaatttttttgagatggagcttttgctgtttcccaggcgggagtgcaatggcgcgctatctcggcgcactgcagcctccatctcctgggttcaagcagttctgcctcagcctcccgagtagcgggattacaggtgcccaccaccacaccctactaattttgtattttagtagagacgacatttcaccatgttggccaggctggctctgaactttgacctcaggtgatccacccaccttaggctctcccaaagtgctaggattacaggtgagatatgctgcgcccggccccaagtgatctattcttgccatgactgttaactaaacatggtgacaggattcgattttctttacattagatttgaaaaccgatgttggttttgggagattgctgcaatttttaggtgacttctctttcagactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaaggtacttaaattagattctgacatacgacatgagttatgtttaaaggaggcacttaagtgtttgtggctactgatgtgtgatacattgtttgacatcttgtccagagcctgtcaaagaagcacctggaaaacgaaagaaggaaatggccaaacagaaagcagctcctgaagccaagaaacagaaagtggaaggtaacttgcagaattaggggatatgggggagataaacagcacaaatgatgaataacaaagggacttaatactgaaaccagatgttacattgtagtgtgctgatgtgctgtgtatagaaattttgctttggaaactaactttttaccacactacaagtagactgagttgagctttttttgtgcaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggtagctgcttcactgcacgttacataccgtgggtctgttaatttttccttcccctgttagcacagttactttagcctgccactgttaaacatgaatactgtaaacacttcaaggttagcattagtgaactaagttagaattaaactgtagatcccctaagttgcaatttccataatcagtcgtaacttggtatagcacagaataatttttagtaatttttttgttgtttttgttatgtattgagacggacgctggcttttgttcaggctggagtacagtggcgcaatcttggctcactgcaacctctgcctcccgggttcaagcgattctcctgcctaacctcccaagtgactgggatacgggtgccactcaccatgcatggctaatttttgttttgtatttagtatcgatttcaccatgttggtcggctggttttgaactcctgacctcaagtgatccacccacctcggcctctcgaagtgctggtacagcgtcaccaccctgccagtaagttttaataatttggtgttaggtgggagaatgcttgaacctgggaggcagaggttgcagtgagccaagttcgcgccactgtactccagcctgggcaacagattgagacaccgtctcaatttaaaataatgtttattttcttggaagtaccttgaaactattagacctgtctagtcatcatagtgaatacttttatccagacaggattctcctgtattagtgcttataggtgttcttttgtcagctgctactgtgaattcttataagcaatttagctccatgatgaagacctcaaacgtgaatgtgcatgtcatatcttcatgctgagccgtgttctgtagctgcagtttgcagagccttgactttgttttgctatactaggggtgctttttaaaatgtgatctttgtttgcaccatcacatttgtctagatacagattgtgattttgatttgtgttttcacctgttgtaattttgccctcctctccacctgaaggaaatttggttatgtggattttgaatctgctgaagacctggagaaagcgttggaactcactggtttgaaagtctttggcaatgaaattaaactagagaaaccaaaaggaaaagacagtaagaaaggtatgtaaggctttatgagttatgcaatgaactcaggagctagactgctagggaaaatgctttgtaacccatttccctttggtttcctcttattttttttaaatcatttttttcctttggtttcctcttaatgtgggaattaaatgagctacagtgtttacaaggtacttggcactgcttgtcagtgtataggtaaattcctgagttaggcaagcaagagcactcttatacagaacaagaaccattacatgcacctaaattaagctaaggatctttcttcactgaaactagttaggtccctaattactccctatatacagtgtaatgttttgaattggtacattcactttttttgttatgcgcgtctactctaggttgaactccagtgtacctaacagagagtttgacatcaaggctgtgacaacatggagggaccacttgtgtgttgacactgctatatctccatatttagcaccgagccttgtacatataggatctcaaattatttgttgatagagctatgtgtgtttttcccctctttttgttgttgccccccacctttggtttttcaggccacagagctcatttttgtttttttaatctagagcgagatgcgagaacacttttggctaaaaatctcccttacaaagtcactcaggatgaattgaaagaagtgtttgaagatgctgcggagatcagattagtcagcaaggatgggaaaagtaaagggtatgttcttctattgaaatgtaagggttttattaacattaatgcacttcctgctttataaaagaaatattggtttgatttccttaggcgtgtaacttggacagtttaacctgtaagtttgtgcctcagtaacccatctgtaccatggggataatgtactcatagggtgattttaaaagacaaagctaatacttacaaagaagcaagtttaatgcctatcttacataaatactttgtaagtagtagcagttctttcagtgaggtgaggttacatgaaaaaattccaagtatttgtaaaactagtgggaagtaagagggaagctcgagttttgattgaaaagtggactaaacaagggcattttatgtactcagatctgaagcaagttctgtgttgctgaggtaaaagcatttgtgttaatatggttttaaaaaccatgagttcttctccctccattgcaggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtaagaaattaggcttgttccaaggttttcagaattggttgagggaactcttctagtctttgtatttcataagtttataaatactttttaatcaaagttactcaaatgtaggtgaagatcaaggacatgataccccaagtcatactcttatttggaatagtaatttccaatcttgaaatgagagctctaaatcattttgcattggaatacagtaggcaaatcaagcttcctttgtaggcatgttttatactttaaatgacttgaccatgtgcgttttgaactcagatgattctaggaaaacagaccagtcatcagcctatgtaagaacaaccagcaggacattgcaacacgtactaggtacttaatatgttgagtaacagaaatggatttagcttacgtcatgagtatttgtatataactcaagcactgaaattcttagggaatagatattactgttgtgaccgaagctgggacactgtttcagagtcttaggaatgtggctctctatttcgaggtgaatcaaaaactctggttttaagcaacctctcctacagtgcaacagaagaaactcttcaggaagtatttgagaaagcaacttttatcaaagtaccccagaaccaaaatggcaaatctaaagggtaagataatacctttgtatcatcagttataggcctatatatgtcttagaggtctaaggacgtaaggtcatgtgtcctgtagaaaaaagctaaataattttagcctagtaaatgagtgtaaaataagtatatttaggtccaaccttgagagaagggccttggccagatcatgtgaccagtggtatagagagcatgtgcctggtaaattactctaagcattaactgttcatcctcaggtatgcatttatagagtttgcttcattcgaagacgctaaagaagctttaaattcctgtaataaaagggaaattgagggcagagcaatcaggctggagttgcaaggacccaggggatcacctaatgccagaagccgtaagttcacctggttagggtgctgtggttgggggtagcactctcggtgctttgtttatttttgcacaaattctgtgtttcctgttcgctactgagtgaacaataactggatatcgatgactgattacctgagaaataattgatgaaatctcaagaaaattcctctagatagtcaagttctgatccagctgtcgtcaactcagagtagcaagtttgcccatgatttcctgccccatccactgggccccacctgcttgggttgctttcccactttccatagaagactggggcaggatatcaactatgcaatggcaattaaaaaatgtaaacccagaatagcctttactttaattaaggactagttggcttagttgcttttaactgctttttcactataacaagtatcttggctagtagtcatactaggcattgtgcaaattcagtgtacgaactgtgaattcacataaatcgcaaatttttttttccttcccagagccatccaaaactctgtttgtcaaaggcctgtctgaggataccactgaagagacattaaaggagtcatttgacggctccgttcgggcaaggatagttactgaccgggaaactgggtcctccaaagggtaagggaaggaagcgtgagtgctgcttccacttgaaggggtttttgttctgtgcagaccttgagtctaatgtgtcttctcattgagctccttctgtctatcagtggcagtttatggattcgcacgagaagaagagagaattcacagaactagcattattttaccttctgtctttacagaggtatatttagctgtattgtgagacattctggggttcaagctgtcacaccagttagttttccatagagagctactctgctgcactggtatctttttcccaaataaacaaggctacttctgtgggatggctccccagcatgtacagttaacttgggacatgtgtagtaggtgctttttataatgggcaatttcatttggtgttctaggtttggttttgtagacttcaacagtgaggaggatgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggaggtaaggcacgcagagataatgacaccacatagcatgtgctcttcagaccctgtgccctgtcacggttcctaatcactggggaggaggagctttgtacccattcttttaacagtgtcttgccttcctcctgtagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgttacttttttttttgtttttgttttttttttttttgcgttcgtggggttgtaaaagaaaagaaagcagaatgttttatcatggtttttgcttcaccgctttaggacaaattaaaagtcaactctggtgccagacgtgttacttcctaaagagtgtttcccctggaatctcactggagagcatggcaaagccagctctgccacttgcttcacccatcccaatggaaatggcttagtgcgtgtttccagtatcccagccctaactaacttggttgaaatgctggtgaggggacctgctcctgcagccctggtgctgacttgaaggctgctgcagcttctcctacttttagcaggtctcgaggattatgtctgaagaccactctggaaagaggtcgaggaacagattagtcaggtttcctagg

SEQ ID NO: 5 - DNA, 2723 bp, Homo sapiens
aggctcagtctttcgcctcagtctcgagctctcgctggccttcgggtgtacgtgctccgggatcttcagcacccgcggccgccatcgccgtcgcttggcttcttctggactcatctgcgccacttgtccgcttcacactccgccgccatcatggtgaagctcgcgaaggcaggtaaaaatcaaggtgaccccaagaaaatggctcctcctccaaaggaggtagaagaagatagtgaagatgaggaaatgtcagaagatgaagaagatgatagcagtggagaagaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatgactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaagagcctgtcaaagaagcacctggaaaacgaaagaaggaaatggccaaacagaaagcagctcctgaagccaagaaacagaaagtggaaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggaaatttggttatgtggattttgaatctgctgaagacctggagaaagcgttggaactcactggtttgaaagtctttggcaatgaaattaaactagagaaaccaaaaggaaaagacagtaagaaagagcgagatgcgagaacacttttggctaaaaatctcccttacaaagtcactcaggatgaattgaaagaagtgtttgaagatgctgcggagatcagattagtcagcaaggatgggaaaagtaaagggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtgaatcaaaaactctggttttaagcaacctctcctacagtgcaacagaagaaactcttcaggaagtatttgagaaagcaacttttatcaaagtaccccagaaccaaaatggcaaatctaaagggtatgcatttatagagtttgcttcattcgaagacgctaaagaagctttaaattcctgtaataaaagggaaattgagggcagagcaatcaggctggagttgcaaggacccaggggatcacctaatgccagaagccagccatccaaaactctgtttgtcaaaggcctgtctgaggataccactgaagagacattaaaggagtcatttgacggctccgttcgggcaaggatagttactgaccgggaaactgggtcctccaaagggtttggttttgtagacttcaacagtgaggaggatgccaaagctgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgtttcttttttttgttttgtttttgttttttttttttttgcgttcgtggggttgtaaaagaaaagaaagcagaatgttttatcatggtttttgcttcagcggctttaggacaaattaaaagtcaactctggtgcc

SEQ ID NO: 6 - protein, 14 aa, Artificial Sequence
  Description of Artificial Sequence Synthetic peptide
RRWCYRKKPYRKCR

SEQ ID NO: 7 - DNA, 31 bp, Artificial Sequence
  Description of Artificial Sequence Primer
ggggatccatggtgaagctcgcgaaggcagg

SEQ ID NO: 8 - DNA, 31 bp, Artificial Sequence
  Description of Artificial Sequence Primer
ccgaattcttctttgacaggctcttcctcct

SEQ ID NO: 9 - DNA, 29 bp, Artificial Sequence
  Description of Artificial Sequence Primer
ggggatccgaagcacctggaaaacgaaag

SEQ ID NO: 10 - DNA, 35 bp, Artificial Sequence
  Description of Artificial Sequence Primer
gggaattcctattcaaacttcgtcttctttccttg

SEQ ID NO: 11 - DNA, 30 bp, Artificial Sequence
  Description of Artificial Sequence Primer
ggatccaatctctttgttggaaacctaaac

SEQ ID NO: 12 - DNA, 30 bp, Artificial Sequence
  Description of Artificial Sequence Primer
ggatccacacttttggctaaaaatctccct

SEQ ID NO: 13 - DNA, 30 bp, Artificial Sequence
  Description of Artificial Sequence Primer
gaattcttttgattcaccactccaagtgct

SEQ ID NO: 14 - DNA, 30 bp, Artificial Sequence
  Description of Artificial Sequence Primer
ggatccactctggttttaagcaacctctcc

SEQ ID NO: 15 - DNA, 30 bp, Artificial Sequence
  Description of Artificial Sequence Primer
gaattctttggatggctggcttctggcatt

SEQ ID NO: 16 - DNA, 30 bp, Artificial Sequence
  Description of Artificial Sequence Primer
ggatccactctgtttgtcaaaggcctgtct

SEQ ID NO: 17 - DNA, 30 bp, Artificial Sequence
  Description of Artificial Sequence Primer
gaattcaggtttggcccagtccaaggtaac

SEQ ID NO: 18 - DNA, 30 bp, Artificial Sequence
  Description of Artificial Sequence Primer
ggatccaagggtgaaggtggcttcgggggt

SEQ ID NO: 19 - DNA, 30 bp, Artificial Sequence
  Description of Artificial Sequence Primer
gaattcctattcaaacttcgtcttctttcc

SEQ ID NO: 20 - protein, 12 aa, Homo sapiens
DHKPQGKKTKFE

SEQ ID NO: 21 - protein, 50 aa, Artificial Sequence
  Description of Artificial Sequence Synthetic peptide
KGEGGFGGRGGGRGGFGGRGGGRGGRGGFGGRGRGGFGGRGGFRGGRGGG

SEQ ID NO: 22 - protein, 40 aa, Artificial Sequence
  Description of Artificial Sequence Synthetic peptide
GGRGGFGGRGRGGFGGRGGFRGGRGGGGDHKPQGKKTKFE

SEQ ID NO: 23 - protein, 25 aa, Artificial Sequence
  Description of Artificial Sequence Synthetic peptide
GRGGFGGRGRGGFGGRGGFRGGRGG

SEQ ID NO: 24 - protein, 16 aa, Artificial Sequence
  Description of Artificial Sequence Synthetic peptide
GGGGDHKPQGKKTKFE

SEQ ID NO: 25 - protein, 15 aa, Artificial Sequence
  Description of Artificial Sequence Synthetic peptide
GRGGFGGRGGFGGRG